(19) United States
(12) Patent Application Publication
(10) Pub. No.: US 2005/0171230 A1
(43) Pub. Date: Aug. 4, 2005

(54) Agent for suppressing transfer of odor and taste originating from a diacetal a diacetal composition comprising the agent for suppressing transfer of odor and taste a polyolefin nucleating agent comprising the composition a polyolefin resin composition and a molded product comprising the nucleating agent

(76) Inventors: Masahide Ishikawa, Kyoto (JP); Chiaki Ueoka, Nara-shi Nara (JP)

Correspondence Address: ARMSTRONG, KRATZ, QUINTOS, HANSON & BROOKS, LLP, 1725 K STREET, NW, SUITE 1000, WASHINGTON, DC, 20006, US

(21) Appl. No.: 10/500,867

(22) PCT Filed: Apr. 28, 2003
(86) PCT No.: PCT/JP03/05424

(30) Foreign Application Priority Data
May 2, 2002 (JP) 2002-130873

(51) Int. Cl. A61L 2/00

(52) U.S. Cl. 523/102

(57) ABSTRACT

Disclosed are an agent for suppressing transfer of odor and taste originating from a diacetal such as dibenzylidene sorbitol, comprising (B) an aliphatic alcohol or an aliphatic carboxylic acid containing at least one hydroxyl group, or comprising component (B) and (C) at least one member selected from the group consisting of specific sulfuric ester salts, saturated or unsaturated fatty acid alkali metal salts and aliphatic amines; a diacetal composition comprising the suppressing agent and the diacetal; a resin composition and a molded product comprising the diacetal composition and a polyolefin resin; and a method for suppressing transfer of odor and taste originating from the diacetal, or a method for suppressing aldehyde generation by thermal decomposition of the diacetal, comprising using the suppressing agent.

### TECHNICAL FIELD

The present invention relates to an agent for suppressing transfer of odor and taste originating from a diacetal, a diacetal composition comprising the suppressing agent, a polyolefin nucleating agent comprising the diacetal composition, a polyolefin resin composition comprising the nucleating agent, a polyolefin resin molded product prepared by molding the resin composition, and a method for suppressing transfer of odor and taste originating from a diacetal using the suppressing agent.

### BACKGROUND ART

Diacetals such as dibenzylidene sorbitol and nuclear-substituted derivatives thereof are useful compounds used as nucleating agents for polyolefin resins, specifically for homopolymers of ethylene or propylene, and copolymers predominantly comprising ethylene and propylene. Diacetals are especially effective in improving clarity, and widely used as resin additives in the field of molded products such as various containers requiring clarity.

However, the diacetals partly undergo thermal decomposition during processing, and release benzaldehydes constituting the diacetals to thereby emit an odor. For this reason, the aldehyde odor sometimes remains also in the final molded product. Moreover, when a molded product is used as a packaging material or a container for food or the like, a taste of aldehyde may transfer to the food or the like in contact with this molded product. Therefore, the diacetals may not be preferred in the field of containers and packaging materials for foods, cosmetics and the like.

There have been various proposals up to now aimed at ameliorating the above problems. Examples are treatment by a hydroxyamine or a phenylhydrazine (Japanese Unexamined Patent Publication No. 1985-32791 and Japanese Unexamined Patent Publication No. 1985-42385), addition of a non-aromatic organic amine (Japanese Unexamined Patent Publication No. 1987-4289), surface treatment by an aliphatic metal salt, a lactic acid metal salt or the like (Japanese Unexamined Patent Publication No. 1987-50355), addition of an aliphatic amine (Japanese Unexamined Patent Publication No. 1990-1906841), addition of sorbic acid and/or potassium sorbate (Japanese Unexamined Patent Publication No. 1993-202055), addition of an alkali metal salt of an amino acid (Japanese Unexamined Patent Publication No. 1997-286787) and like methods.

Nevertheless, the above prior art methods still have some drawbacks: the addition of said additives might cause yellowing of the polyolefin resin molded product; odor-improving effect is insufficient and limits application in the field of, e.g., food containers; and so on, and these and other problems have yet to be satisfactorily solved.

An object of the present invention is to provide a novel and useful polyolefin resin composition, in which aldehyde generation is significantly suppressed during processing and in the final molded product, thereby suppressing transfer of odor and taste.

### DISCLOSURE OF THE INVENTION

Under the above circumstances, the inventors carried out intensive research to achieve the above object and found that when a diacetal composition comprising a diacetal and a specific long chain fatty alcohol or a specific hydroxycarboxylic acid (component (B)) is used as a nucleating agent for polyolefin, aldehyde generation is significantly suppressed in the resultant polyolefin resin pellets and the molded products thereof, thereby suppressing transfer of odor and taste.

In addition, it was found that a diacetal composition which further contains a specific anionic surfactant, a long chain fatty acid alkali metal salt or an amine (component (C)) in addition to the diacetal composition comprising a diacetal and component (B) achieves a synergetic effect so that aldehyde generation is further suppressed in resin pellets and molded products.

Moreover, there was apprehension that a nucleating agent characteristics of diacetals as a clarifier, especially clarity, might deteriorate by using the above diacetal composition. According to the inventors' research, however, it was found that the clarity of the resulting polyolefin resin molded product was not substantially impaired.

The present invention has been accomplished based on these findings and further researches. The present invention provides the following agent for suppressing transfer of odor and taste originating from a diacetal, diacetal composition, polyolefin nucleating agent, polyolefin resin composition and polyolefin resin molded product and the like.

Item 1. An agent for suppressing transfer of odor and taste originating from (A) a diacetal represented by the formula (1): 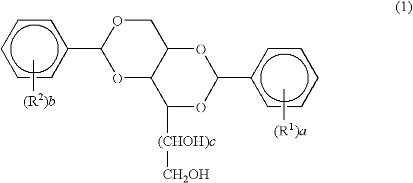
wherein R¹ and R² are the same or different and each represents a hydrogen atom, a C₁ to C₄ alkyl group, a C₁ to C₄ alkoxy group, a C₁ to C₄ alkoxycarbonyl group or a halogen atom; a and b each represents an integer of 1 to 5; c is 0 or 1; when a is 2, the two R¹ groups taken together with the benzene ring to which they are linked may form a tetralin ring; and when b is 2, the two R² groups taken together with the benzene ring to which they are linked may form a tetralin ring;
the agent comprising component (B), i.e., at least one member selected from the group consisting of:
(B1) C₆ to C₃₂ saturated or unsaturated aliphatic alcohols; and
(B2) C₈ to C₃₂ saturated or unsaturated aliphatic carboxylic acids having at least one hydroxyl group per molecule.

Item 2. The agent for suppressing transfer of odor and taste according to item 1, wherein said at least one member selected from the group consisting of (B1) and (B2) is at least one member selected from the group consisting of 9-hydroxystearic acid, 10-hydroxystearic acid, 12-hydroxystearic acid, 9,10-dihydroxystearic acid, lauryl alcohol, myristyl alcohol, palmityl alcohol, stearyl alcohol and behenyl alcohol.

Item 3. A method for suppressing transfer of odor and taste originating from the diacetal represented by the formula (1) according to item 1 or a method for suppressing aldehyde generation by thermal decomposition of the diacetal;

the method comprising adding to the diacetal represented by the formula (1) at least one member selected from the group consisting of:

(B1) C₆ to C₃₂ saturated or unsaturated aliphatic alcohols; and
(B2) C₈ to C₃₂ saturated or unsaturated aliphatic carboxylic acids having at least one hydroxyl group per molecule.

Item 4. The method according to item 3, wherein said at least one member selected from the group consisting of components (B1) and (B2) is: at least one member selected from the group consisting of 9-hydroxystearic acid, 10-hydroxystearic acid, 12-hydroxystearic acid, 9,10-dihydroxystearic acid, lauryl alcohol, myristyl alcohol, palmityl alcohol, stearyl alcohol and behenyl alcohol.

Item 5. An agent for suppressing transfer of odor and taste originating from (A) at least one diacetal represented by the formula (1): 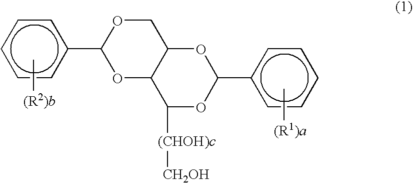
wherein R¹ and R² are the same or different and each represents a hydrogen atom, a C₁ to C₄ alkyl group, a C₁ to C₄ alkoxy group, a C₁ to C₄ alkoxycarbonyl group or a halogen atom; a and b each represents an integer of 1 to 5; c is 0 or 1; when a is 2, the two R¹ groups taken together with the benzene ring to which they are linked may form a tetralin ring; and when b is 2, the two R² groups taken together with the benzene ring to which they are linked may form a tetralin ring;
the agent comprising components (B) and (C),
wherein component (B) is at least one member selected from the group consisting of:
(B1) C₆ to C₃₂ saturated or unsaturated aliphatic alcohols; and
(B2) C₈ to C₃₂ saturated or unsaturated aliphatic carboxylic acids having at least one hydroxyl group per molecule, and component (C) is
(C1) at least one anionic surfactant selected from the group consisting of C₆ to C₃₀ saturated or unsaturated aliphatic alcohol sulfuric ester salts, polyoxyethylene alkyl (C₈ to C₂₂) or alkenyl (C₈ to C₂₂) ether sulfuric ester salts in which the number of moles of ethylene oxide added is 1 to 8, polyoxyethylene alkyl (C₈ to C₂₂) phenyl ether sulfuric ester salts in which the number of moles of ethylene oxide added is 1 to 10, sulfuric ester salts of polyhydric alcohol fatty acid partial esters formed from a C₃ to C₆ polyhydric alcohol and a C₈ to C₂₂ saturated or unsaturated fatty acid, and C₈ to C₂₂ saturated or unsaturated fatty acid monoalkanol (C₂ to C₆) amide sulfuric ester salts, wherein the sulfuric ester salts are lithium salts, sodium salts, potassium salts and ammonium salts;
(C2) at least one member selected from the group consisting of alkali metal salts of C₈ to C₃₂ saturated or unsaturated fatty acids which may have at least one hydroxyl group per molecule;
(C3) at least one aliphatic amine selected from the group consisting of dialkanolamine, trialkanolamine, and di(C₈ to C₂₂ alkyl or alkenyl) methylamine; or
(C4) a mixture of at least two of (C1), (C2) and (C3).

Item 6. The agent for suppressing transfer of odor and taste according to item 5, wherein
component (B) is at least one member selected from the group consisting of 9-hydroxystearic acid, 10-hydroxystearic acid, 12-hydroxystearic acid, 9,10-dihydroxystearic acid, lauryl alcohol, myristyl alcohol, palmityl alcohol, stearyl alcohol and behenyl alcohol, and
component (C) is (C2a) at least one member selected from the group consisting of lithium salts, sodium salts and potassium salts of C₈ to C₃₂ saturated or unsaturated fatty acids which may have at least one hydroxyl group per molecule, or
component (C) is (C1a) at least one sulfuric ester salt selected from the group consisting of lauryl sulfate salts, stearyl sulfate salts, oleyl sulfate salts, polyoxyethylene (the number of moles of ethylene oxide added=2 to 3) lauryl ether sulfate salts, polyoxyethylene (the number of moles of ethylene oxide added=2 to 3) stearyl ether sulfate salts, polyoxyethylene (the number of moles of ethylene oxide added=2 to 3) nonylphenyl ether sulfate salts, polyoxyethylene (the number of moles of ethylene oxide added=2 to 3) dodecylphenyl ether sulfate salts, glyceryl monolaurate sulfate salts, glyceryl monostearate sulfate salt, lauric acid monoethanolamide sulfuric ester salts, stearic acid monoethanolamide sulfuric ester salts, and oleic acid monoethanolamide sulfuric ester salts, wherein the sulfuric ester salts or sulfate salts are lithium salts, sodium salts and potassium salts.

Item 7. The agent for suppressing transfer of odor and taste according to item 6, wherein component (C) is at least one member selected from the group consisting of sodium lauryl sulfate, potassium lauryl sulfate, sodium stearate, potassium stearate, sodium 12-hydroxystearate and potassium 12-hydroxystearate.

Item 8. The agent for suppressing transfer of odor and taste according to any one of items 5-7, wherein the weight ratio of component (B) to component (C) is 1:0.2 to 5.

Item 9. A method for suppressing transfer of odor and taste originating from (A) at least one diacetal represented by the formula (1): 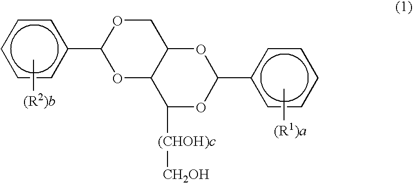
wherein R¹ and R² are the same or different and each represents a hydrogen atom, a C₁ to C₄ alkyl group, a C₁ to C₄ alkoxy group, a C₁ to C₄ alkoxycarbonyl group or a halogen atom; a and b each represents an integer of 1 to 5; c is 0 or 1; when a is 2, the two R¹ groups taken together with the benzene ring to which they are linked may form a tetralin ring; and when b is 2, the two R² groups taken together with the benzene ring to which they are linked may form a tetralin ring
or a method for suppressing aldehyde generation by thermal decomposition of the diacetal;
the method comprising adding the following components (B) and (C) to the diacetal,
wherein component (B) is at least one member selected from the group consisting of:
(B1) C₆ to C₃₂ saturated or unsaturated aliphatic alcohols; and
(B2) C₈ to C₃₂ saturated or unsaturated aliphatic carboxylic acids having at least one hydroxyl group per molecule, and component (C) is
(C1) at least one anionic surfactant selected from the group consisting of C₆ to C₃₀ saturated or unsaturated aliphatic alcohol sulfuric ester salts, polyoxyethylene alkyl (C₈ to C₂₂) or alkenyl (C8 to C₂₂) ether sulfuric ester salts in which the number of moles of ethylene oxide added is 1 to 8, polyoxyethylene alkyl (C₈ to C₂₂) phenyl ether sulfuric ester salts in which the number of moles of ethylene oxide added is 1 to 10, sulfuric ester salts of polyhydric alcohol fatty acid partial esters formed from a C₃ to C₆ polyhydric alcohol and a C₈ to C₂₂ saturated or unsaturated fatty acid, and C₈ to C₂₂ saturated or unsaturated fatty acid monoalkanol (C₂ to C₆) amide sulfuric ester salts, wherein the sulfuric ester salts are lithium salts, sodium salts, potassium salts and ammonium salts;
(C2) at least one member selected from the group consisting of alkali metal salts of C₈ to C₃₂ saturated or unsaturated fatty acids which may have at least one hydroxyl group per molecule;
(C3) at least one aliphatic amine selected from the group consisting of dialkanolamine, trialkanolamine, and di(C₈ to C₂₂ alkyl or alkenyl) methylamine; or
(C4) a mixture of at least two of (C1), (C2) and (C3).

Item 10. The method according to item 9, wherein component (B) is at least one member selected from the group consisting of 9-hydroxystearic acid, 10-hydroxystearic acid, 12-hydroxystearic acid, 9,10-dihydroxystearic acid, lauryl alcohol, myristyl alcohol, palmityl alcohol, stearyl alcohol and behenyl alcohol, and
component (C) is (C2a) at least one member selected from the group consisting of lithium salts, sodium salts and potassium salts of C₈ to C₃₂ saturated or unsaturated fatty acids which may have at least one hydroxyl group per molecule, or
component (C) is (C1a) at least one sulfuric ester salt selected from the group consisting of lauryl sulfate salts, stearyl sulfate salts, oleyl sulfate salts, polyoxyethylene (the number of moles of ethylene oxide added=2 to 3) lauryl ether sulfate salts, polyoxyethylene (the number of moles of ethylene oxide added=2 to 3) stearyl ether sulfate salts, polyoxyethylene (the number of moles of ethylene oxide added=2 to 3) nonylphenyl ether sulfate salts, polyoxyethylene (the number of moles of ethylene oxide added=2 to 3) dodecylphenyl ether sulfate salts, glyceryl monolaurate sulfate salts, glyceryl monostearate sulfate salts, lauric acid monoethanolamide sulfuric ester salts, stearic acid monoethanolamide sulfuric ester salts, and oleic acid monoethanolamide sulfuric ester salts, wherein the sulfuric ester salts or sulfate salts are lithium salts, sodium salts and potassium salts.

Item 11. The method according to item 10, wherein component (C) is at least one member selected from the group consisting of sodium lauryl sulfate, potassium lauryl sulfate, sodium stearate, potassium stearate, sodium 12-hydroxystearate and potassium 12-hydroxystearate.

Item 12. The method according to any one of items 9-11, wherein the weight ratio of component (B) to component (C) is 1:0.2 to 5.

Item 13. A granular or powdery diacetal composition comprising:
(A) at least one diacetal represented by the formula (1) 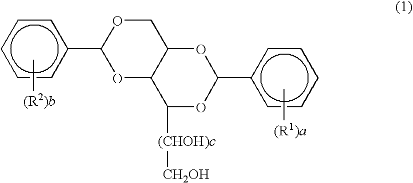
   wherein R¹ and R² are the same or different and each represents a hydrogen atom, a C₁ to C₄ alkyl group, a C₁ to C₄ alkoxy group, a C₁ to C₄ alkoxycarbonyl group or a halogen atom; a and b each represents an integer of 1 to 5; c is 0 or 1; when a is 2, the two R¹ groups taken together with the benzene ring to which they are linked may form a tetralin ring; and when b is 2, the two R² groups taken together with the benzene ring to which they are linked may form a tetralin ring; and
   component (B), i.e., at least one member selected from the group consisting of
   (B1) C₆ to C₃₂ saturated or unsaturated aliphatic alcohols and
   (B2) C₈ to C₃₂ saturated or unsaturated aliphatic carboxylic acids having at least one hydroxyl group per molecule,
      wherein transfer of odor and taste originating from the diacetal is suppressed.

Item 14. The diacetal composition according to item 13, wherein component (B) is present in a proportion of 0.1 to 10 wt % based on the total amount of components (A) and (B).

Item 15. A granular or powdery diacetal composition wherein transfer of odor and taste originating from the diacetal is suppressed;
the composition comprising components (A), (B) and (C), wherein component (A) is at least one diacetal represented by the formula (1) 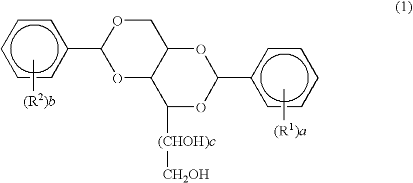
wherein R¹ and R² are the same or different and each represents a hydrogen atom, a C₁ to C₄ alkyl group, a C₁ to C₄ alkoxy group, a C₁ to C₄ alkoxycarbonyl group or a halogen atom; a and b each represents an integer of 1 to 5; c is 0 or 1; when a is 2, the two R¹ groups taken together with the benzene ring to which they are linked may form a tetralin ring; and when b is 2, the two R² groups taken together with the benzene ring to which they are linked may form a tetralin ring,
component (B) is at least one member selected from the group consisting of:
(B1) C₆ to C₃₂ saturated or unsaturated aliphatic alcohols; and
(B2) C₈ to C₃₂ saturated or unsaturated aliphatic carboxylic acids having at least one hydroxyl group per molecule, and
   component (C) is
(C1) at least one anionic surfactant selected from the group consisting of C₆ to C₃₀ saturated or unsaturated aliphatic alcohol sulfuric ester salts, polyoxyethylene alkyl (C₈ to C₂₂) or alkenyl (C₈ to C₂₂) ether sulfuric ester salts in which the number of moles of ethylene oxide added is 1 to 8, polyoxyethylene alkyl (C₈ to C₂₂) phenyl ether sulfuric ester salts in which the number of moles of ethylene oxide added is 1 to 10, sulfuric ester salts of polyhydric alcohol fatty acid partial esters formed from a C₃ to C₆ polyhydric alcohol and a C₈ to C₂₋₂ saturated or unsaturated fatty acid, and C₈ to C₂₂ saturated or unsaturated fatty acid monoalkanol (C₂ to C₆) amide sulfuric ester salts, wherein the sulfuric ester salts are lithium salts, sodium salts, potassium salts and ammonium salts;
(C2) at least one member selected from the group consisting of alkali metal salts of C₈ to C₃₂ saturated or unsaturated fatty acids which may have at least one hydroxyl group per molecule;
(C3) at least one aliphatic amine selected from the group consisting of dialkanolamine, trialkanolamine, and di(C₈ to C₂₂ alkyl or alkenyl) methylamine; or
(C4) a mixture of at least two of (C1), (C2) and (C3).

Item 16. The diacetal composition according to item 15, wherein based on the total amount of components (A), (B) and (C), component (B) is present in a proportion of 0.1 to 5 wt % and component (C) is present in a proportion of 0.1 to 5 wt %.

Item 17. The diacetal composition according to item 16, wherein the weight ratio of component (B) to component (C) is 1:0.2 to 5.

Item 18. A polyolefin resin nucleating agent comprising the diacetal composition according to any one of items 13 to 17, wherein transfer of odor and taste originating from the diacetal is suppressed.

Item 19. A polyolefin resin composition comprising the polyolefin resin nucleating agent according to item 18 and a polyolefin resin, wherein transfer of odor and taste originating from the diacetal is suppressed.

Item 20. The polyolefin resin composition according to item 19, wherein the polyolefin resin nucleating agent according to item 18 is present in an amount of 0.05 to 3 weight parts per 100 weight parts of the polyolefin resin.

Item 21. A polyolefin resin molded product prepared by molding the polyolefin resin composition according to item 19 or 20, wherein transfer of odor and taste originating from the diacetal is suppressed.

Item 22. A container or a packaging material for foods, cosmetics or medicines comprising the polyolefin resin molded product according to item 21, wherein transfer of odor and taste originating from the diacetal is suppressed.

Item 23. A method for suppressing odor originating from a diacetal at the time of molding a polyolefin resin, comprising mixing the nucleating agent according to item 18 with a polyolefin resin and molding a resultant resin composition.

Item 24. A method for suppressing transfer of odor and taste originating from a diacetal to a content (such as foods, cosmetics and medicines), characterized in that it comprises placing the content in a packaging material or a container prepared by mixing the nucleating agent according to item 18 with a polyolefin resin and molding a resultant resin composition.

### DETAILED DESCRIPTION OF THE INVENTION

Component (A): Diacetal

The diacetals of which suppression of odor and taste transfer is contemplated by the present invention are represented by the above formula (1).

In the formula (1), examples of C₁ to C₄ alkoxy groups represented by R¹ and R² include methyl group, ethyl group, propyl group, isopropyl group, butyl group, etc. Examples of C₁ to C₄ alkoxy groups include methoxy group, ethoxy group, propoxy group, isopropoxy group, butoxy group, etc. Examples of C₁ to C₄ alkoxycarbonyl groups include methoxycarbonyl group, ethoxycarobonyl group, propoxycarbonyl group, isopropoxycarbonyl group, etc. Examples of halogen atoms include fluorine atom, chlorine atom, bromine atom, etc. a and b are each an integer of 1 to 5,
preferably 1, 2 or 3. c is preferably 1. There are no particular restrictions on the position of the substituents represented by R¹ and R², but examples include o-, m- and p-positions when a and b are each 1, and 2,4-, 3,4- and 3,5-positions when a and b are each 2, or 2,4,5- and 3,4,5-positions when a and b are each 3.

All of the diacetals represented by the formula (1) above are known or can be readily prepared by a known process, such as those set forth in Japanese Examined Patent Publication S48-43748 and Japanese Unexamined Patent Publications Nos. S53-5165, S57-185287 and H2-231488.

The following are typical examples of the diacetal represented by the formula (1).

1,3:2,4-O-dibenzylidene-D-sorbitol, 1,3:2,4-bis-O-(o-methylbenzylidene)sorbitol, 1,3:2,4-bis-O-(m-methylbenzylidene)-D-sorbitol, 1,3:2,4-bis-O-(m-ethylbenzylidene)-D-sorbitol, 1,3:2,4-bis-O-(m-isopropylbenzylidene)-D-sorbitol, 1,3:2,4-bis-O-(m-n-propylbenzylidene)-D-sorbitol, 1,3:2,4-bis-O-(m-n-butylbenzylidene)-D-sorbitol, 1,3:2,4-bis-O-(p-methylbenzylidene)-D-sorbitol, 1,3:2,4-bis-O-(p-ethylbenzylidene)-D-sorbitol, 1,3:2,4-bis-O-(p-isopropylbenzylidene)-D-sorbitol, 1,3:2,4-bis-O-(p-n-propylbenzylidene)-D-sorbitol, 1,3:2,4-bis-O-(p-n-butylbenzylidene)-D-sorbitol, 1,3:2,4-bis-O-(2,3-dimethylbenzylidene)-D-sorbitol, 1,3:2,4-bis-O-(2,4-dimethylbenzylidene)-D-sorbitol, 1,3:2,4-bis-O-(2,5-dimethylbenzylidene)-D-sorbitol, 1,3:2,4-bis-O-(3,4-dimethylbenzylidene)-D-sorbitol, 1,3:2,4-bis-O-(3,5-dimethylbenzylidene)-D-sorbitol, 1,3:2,4-bis-O-(2,3-diethylbenzylidene)-D-sorbitol, 1,3:2,4-bis-O-(2,4-diethylbenzylidene)-D-sorbitol, 1,3:2,4-bis-O-(2,5-diethylbenzylidene)-D-sorbitol, 1,3:2,4-bis-O-(3,4-diethylbenzylidene)-D-sorbitol, 1,3:2,4-bis-O-(3,5-diethylbenzylidene)-D-sorbitol, 1,3:2,4-bis-O-(2,4,5-trimethylbenzylidene)-D-sorbitol, 1,3:2,4-bis-O-(3,4,5-trimethylbenzylidene)-D-sorbitol, 1,3:2,4-bis-O-(2,4,5-triethylbenzylidene)-D-sorbitol, 1,3:2,4-bis-O-(3,4,5-triethylbenzylidene)-D-sorbitol, 1,3:2,4-bis-O-(p-methyloxycarbonylbenzylidene)-D-sorbitol, 1,3:2,4-bis-O-(p-ethyloxycarbonylbenzylidene)-D-sorbitol, 1,3:2,4-bis-O-(p-isopropyloxycarbonylbenzylidene)-D-sorbitol, 1,3:2,4-bis-O-(o-n-propyloxycarbonylbenzylidene)-D-sorbitol, 1,3:2,4-bis-O-(o-n-butylbenzylidene)-D-sorbitol, 1,3:2,4-bis-O-(o-chlorobenzylidene)-D-sorbitol, 1,3:2,4-bis-O-(p-chlorobenzylidene)-D-sorbitol, 1,3:2,4-bis-O-[(5,6,7,8-tetrahydro-1-naphthalene)-1-methylene)]-D-sorbitol, 1,3:2,4-bis-O-[(5,6,7,8-tetrahydro-2-naphthalene)-1-methylene]-D-sorbitol, 1,3-O-benzylidene-2,4-O-p-methylbenzylidene-D-sorbitol, 1,3-O-p-methylbenzylidene-2,4-O-benzylidene-D-sorbitol, 1,3-O-benzylidene-2,4-O-p-ethylbenzylidene-D-sorbitol, 1,3-O-p-ethylbenzylidene-2,4-O-benzylidene-D-sorbitol, 1,3-O-benzylidene-2,4-O-p-chlorobenzylidene-D-sorbitol, 1,3-O-p-chlorobenzylidene-2,4-O-benzylidene-D-sorbitol, 1,3-O-benzylidene-2,4-O-(2,4-dimethylbenzylidene)-D-sorbitol, 1,3-O-(2,4-dimethylbenzylidene)-2,4-O-benzylidene-D-sorbitol, 1,3-O-benzylidene-2,4-O-(3,4-dimethylbenzylidene)-D-sorbitol, 1,3-O-(3,4-dimethylbenzylidene)-2,4-O-benzylidene-D-sorbitol, 1,3-O-p-methyl-benzylidene-2,4-O-p-ethylbenzylidene sorbitol, 1,3-p-ethyl-benzylidene-2,4-p-methylbenzylidene-D-sorbitol, 1,3-O-p-methyl-benzylidene-2,4-O-p-chlorobenzylidene-D-sorbitol, and 1,3-O-p-chloro-benzylidene-2,4-O-p-methylbenzylidene-D-sorbitol. These can be used singly or at least two of them may be used as suitably combined.

Among them, preferable are more effective compounds such as 1,3:2,4-O-dibenzylidene-D-sorbitol, 1,3:2,4-bis-O-(o-methylbenzylidene)sorbitol, 1,3:2,4-bis-O-(p-methylbenzylidene)-D-sorbitol, 1,3:2,4-bis-O-(p-ethylbenzylidene)-D-sorbitol, 1,3:2,4-bis-O-(p-isopropylbenzylidene)-D-sorbitol, 1,3:2,4-bis-O-(p-n-propylbenzylidene)-D-sorbitol, 1,3:2,4-bis-O-(p-n-butylbenzylidene)-D-sorbitol, 1,3:2,4-bis-O-(2,4-dimethylbenzylidene)-D-sorbitol, 1,3:2,4-bis-O-(3,4-dimethylbenzylidene)-D-sorbitol, 1,3:2,4-bis-O-(3,5-dimethylbenzylidene)-D-sorbitol, 1,3:2,4-bis-O-(2,4,5-trimethylbenzylidene)-D-sorbitol, 1,3:2,4-bis-O-(p-methyloxycarbonylbenzylidene)-D-sorbitol, 1,3:2,4-bis-O-[(5,6,7,8-tetrahydro-1-naphthalene)-1-methylene)]-D-sorbitol, 1,3:2,4-bis-O-[(5,6,7,8-tetrahydro-2-naphthalene)-1-methylene]-D-sorbitol, 1,3-O-benzylidene-2,4-O-p-methylbenzylidene-D-sorbitol, 1,3-O-p-methylbenzylidene-2,4-O-benzylidene-D-sorbitol, 1,3-O-benzylidene-2,4-O-(2,4-dimethylbenzylidene)-D-sorbitol, 1,3-O-(2,4-dimethylbenzylidene)-2,4-O-benzylidene-D-sorbitol, 1,3-O-benzylidene-2,4-O-(3,4-dimethylbenzylidene)-D-sorbitol, and 1,3-O-(3,4-dimethylbenzylidene)-2,4-O-benzylidene-D-sorbitol. These may be used singly or at least two of them may be used as suitably combined.

Among them, 1,3:2,4-O-dibenzylidene-D-sorbitol, 1,3:2,4-bis-O-(p-methylbenzylidene)-D-sorbitol, 1,3:2,4-bis-O-(p-ethylbenzylidene)-D-sorbitol, and 1,3:2,4-bis-O-(3,4-dimethylbenzylidene)-D-sorbitol are particularly preferable.

There are no particular restrictions on the crystal form of the diacetal as long as the effect of the invention can be achieved, and any crystal form can be used, such as hexagonal, monoclinic, cubic, trigonal and orthorhombic. These crystals are known or can be manufactured by a known method.

The diacetal used in the present invention may be one in which the purity of the 1,3:2,4-compound represented by the formula (1) is 100%, but may also be one containing a small amount of impurities.

Agent for Suppressing Transfer of Odor and Taste

When a nucleating agent comprising the diacetal represented by the formula (1) is added to a polyolefin resin to obtain pellets or resin composition, and a resin molded product prepared from the resin composition is used as a container or a packaging material for foods, cosmetics or other products, an odor is emitted due to an aldehyde released from the diacetal during production process of the resin molded product. In addition, when foods, cosmetics or other products are contained in the container or the packaging material prepared from the molded product, odor and taste will transfer to these products. An object of the present invention is to provide a method for suppressing aldehyde generation by thermal decomposition of the diacetal (method for reducing an amount of aldehyde generation) or to suppress transfer of the odor and taste from the molded product to foods, cosmetics or other products.

To this end, the following component (B) alone or components (B) and (C) are used as an agent for suppressing odor and taste transfer in the present invention.

Component (B)

Component (B) used in the present invention is at least one member selected from the group consisting of: (B1) C₆ to C₃₂ saturated or unsaturated aliphatic alcohols; and (B2) C₈ to C₃₂ saturated or unsaturated aliphatic carboxylic acids having at least one hydroxyl group per molecule.

Examples of saturated or unsaturated aliphatic alcohols (B1) include C₆ to C₃₂, preferably C₁₀ to C₂₂, saturated or unsaturated aliphatic alcohols. Specific examples are hexanol, octanol, lauryl alcohol, myristyl alcohol, palmityl alcohol, stearyl alcohol, isostearyl alcohol, behenyl alcohol, oleyl alcohol and the like. Among them, lauryl alcohol, myristyl alcohol, palmityl alcohol, stearyl alcohol and behenyl alcohol are recommended.

Examples of saturated or unsaturated aliphatic monocarboxylic acids having at least one hydroxyl group per molecule (B2) include C₈ to C₃₂, preferably C₁₂ to C₂₂, aliphatic monocarboxylic acids having at least one, especially one to two, hydroxyl groups per molecule. Specific examples are 9-hydroxystearic acid, 10-hydroxystearic acid, 12-hydroxystearic acid, 9,10-dihydroxystearic acid. Among them, 12-hydroxystearic acid is recommended. These may be used singly or at least two of them may be used as suitably combined.

Component (C)

Examples of component (C) used together with component (B) in the present invention are the following (C1), (C2), (C3) and a mixture thereof (C4).
(C1): at least one anionic surfactant selected from the group consisting of C₆ to C₃₀ saturated or unsaturated aliphatic alcohol sulfuric ester salts, polyoxyethylene alkyl (C₈ to C₂₂) or alkenyl (C₈ to C₂₂) ether sulfuric ester salts in which the number of moles of ethylene oxide added is 1 to 8, polyoxyethylene alkyl (C₈ to C₂₂) phenyl ether sulfuric ester salts in which the number of moles of ethylene oxide added is 1 to 10, sulfuric ester salts of polyhydric alcohol fatty acid partial esters formed from a C₃ to C₆ polyhydric alcohol and a C₈ to C₂₂ saturated or unsaturated fatty acid, and C₈ to C₂₂ saturated or unsaturated fatty acid monoalkanol (C₂ to C₆) amide sulfuric ester salts, wherein the sulfuric ester salts are lithium salts, sodium salts, potassium salts and ammonium salts,
(C2): at least one member selected from the group consisting of alkali metal salts of CB to C₃₋₂ saturated or unsaturated fatty acids which may have at least one hydroxyl group per molecule,
(C3): at least one aliphatic amine selected from the group consisting of dialkanolamine, trialkanolamine, and di(C₈ to C₂₂ alkyl or alkenyl) methylamine, or
(C4): a mixture of at least two of (C1), (C2) and (C3).

Examples of sulfuric ester salt (C1) used in the present invention include C₆ to C₃₀, preferably C₁₀ to C₂₀, saturated or unsaturated aliphatic alcohol sulfuric ester salts; polyoxyethylene alkyl (C₈ to C₂₂, preferably C₁₀ to C₂₂) or alkenyl (C₈ to C₂₂, preferably C₁₀ to C₂₂) ether sulfuric ester salts in which the number of moles of ethylene oxide added is 1 to 8, preferably 2 to 5; polyoxyethylene alkyl (C₈ to C₂₂, preferably C₉ to C₂₀) phenyl ether sulfuric ester salts in which the number of moles of ethylene oxide added is 1 to 10, preferably 2 to 5; sulfuric ester salts of polyhydric alcohol fatty acid partial esters formed from a C₃ to C₆, preferably C₃ or C₄, polyhydric alcohol and a C₈ to C₂₂, preferably C₁₀ to C₂₀, saturated or unsaturated fatty acid; and C₈ to C₂₂, preferably C₁₀ to C₂₀, saturated or unsaturated fatty acid alkanol(C₂ to C₆, preferably C₂ to C₄) amide sulfuric ester salts. The sulfuric ester salt is a lithium salt, sodium salt, potassium salt, or ammonium salt.

Among the above sulfuric ester salts (C1), those represented by the following formulae (a), (b) and (c) are preferred.

A saturated or unsaturated aliphatic alcohol sulfuric ester salt represented by the formula (a):

R^{a}—OSO₃M (a)

wherein R^{a} is a C₆ to C₃₀, preferably C₁₀ to C₂₀, saturated or unsaturated aliphatic group (particularly an alkyl or alkenyl group), and M is Li, Na, K or NH₄.

A polyoxyethylene alkyl or alkenyl ether sulfuric ester salt represented by the formula (b):
R^{b}O—(CH₂CH₂O)ₘ—SO₃M (b)
wherein R^{b} is an alkyl group (C₈ to C₂₂, preferably C₁₀ to C₂₂) or alkenyl group (C₈ to C₂₂, preferably C₁₀ to C₂₂), m is an integer of 1 to 8, preferably 2 to 5, and M is Li, Na, K or NH₄.

A polyoxyethylene alkylphenyl ether sulfuric ester salt represented by the formula (c): 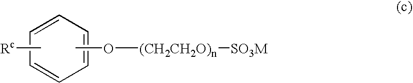
wherein R^{c} is an alkyl group (C₈ to C₂₂, preferably C₉ to C₂₀), n is an integer of 1 to 10, preferably 2 to 5, and M is Li, Na, K or NH₄.

Specific examples of component (C1) include sodium lauryl sulfate, sodium stearyl sulfate, sodium oleyl sulfate, sodium polyoxyethylene (the number of moles of ethylene oxide added=2 to 3) lauryl ether sulfate, sodium polyoxyethylene (the number of moles of ethylene oxide added=2 to 3) stearyl ether sulfate, sodium polyoxyethylene (the number of moles of ethylene oxide added=2 to 3) nonylphenyl ether sulfate, sodium polyoxyethylene (the number of moles of ethylene oxide added=2 to 3) dodecylphenyl ether sulfate, sodium glyceryl monolaurate sulfate, sodium glyceryl monostearate sulfate, sodium lauric acid monoethanolamide sulfate, sodium stearic acid monoethanolamide sulfate, and sodium oleic acid monoethanolamide sulfate.

Preferred examples include sodium lauryl sulfate, sodium stearyl sulfate, sodium oleyl sulfate, sodium polyoxyethylene (the number of moles of ethylene oxide added=2 to 3) lauryl ether sulfate, sodium polyoxyethylene (the number of moles of ethylene oxide added=2 to 3) stearyl ether sulfate, sodium polyoxy (the number of moles of ethylene oxide added=2 to 3) nonylphenyl ether sulfate, and sodium polyoxy (the number of moles of ethylene oxide added=2 to 3) dodecylphenyl ether sulfate.

In addition to the above sodium salts, other examples of the above-mentioned sulfuric ester salts include lithium salts, potassium salts, and ammonium salts. The above-mentioned sulfuric ester salts are lithium salts, sodium salts, potassium salts or ammonium salts, among which lithium salts, sodium salts or potassium salts are recommended. The anionic surfactants listed above can be used singly or at least two of them may be used as suitably combined.

Examples of the alkali metal salts of saturated or unsaturated fatty acids (C2) which may have at least one hydroxyl group per molecule include metal salts of C₈ to C₃₂, preferably C₁₀ to C₂₂ saturated or unsaturated fatty acids that may have at least one (particularly one or two, especially one) hydroxyl group per molecule. These can be used singly or at least two of them may be used as suitably combined.

Specific examples are lithium salts, sodium salts, potassium salts, rubidium salts and cesium salts of octanoic acid, capric acid, lauric acid, myristic acid, palmitic acid, stearic acid, 12-hydroxystearic acid, behenic acid, montanic acid, oleic acid, linoleic acid, linolenic acid, eleostearic acid, ricinoleic acid and erucic acid. Among them, lithium salts, sodium salts and potassium salts of lauric acid, myristic acid, palmitic acid, stearic acid, and 12-hydroxystearic acid are particularly preferred.

Examples of the aliphatic amine (C3) used in the invention include di- or tri-alkanolamines, particularly di- or tri-(C₁ to C₄ alkanol)amines such as diethanolamine, dipropanolamine, diisopropanolamine, butanol amine, triethanolamine, tripropanolamine, triisopropanolamine, tributanolamine and the like, and di(C₈ to C₂₂ alkyl or alkenyl)methylamines such as distearylmethylamine, dihexadecylmethylamine, ditetradecylmethylamlne, didodecylmethylamine, dioleoylmethylamine, stearyllaurylmethylamine and the like. Among them, diethanolamine and disopropanolamine are preferred. These can be used singly or at least two of them may be used as suitably combined.

It is more preferable that component (C) is at least one member selected from the group consisting of sodium lauryl sulfate, potassium lauryl sulfate, sodium stearate, potassium stearate, sodium 12-hydroxystearate and potassium 12-hydroxystearate.

In the present invention, either component (B) singly or a combination of components (B) and (C) may be used as an agent for suppressing transfer of odor and taste originating from a diacetal as component (A).

In the case component (B) is singly used, the amount of component (B) used in the present invention is in the range of 0.1 to 10 wt %, preferably 1 to 5 wt %, based on the total amount of a diacetal as component (A) and component (B). If the amount of component (B) is less than 0.1 wt %, the effect of suppressing aldehyde generation is so weak that odor and taste evaluations show a tendency to be adversely affected. On the other hand, if the amount of component (B) is more than 10 wt %, it is rather difficult to give further effect of suppressing aldehyde generation, and nucleating agent characteristics as a clarifier of polyolefin resins tend to deteriorate.

When components (B) and (C) are jointly used, the amount of component (B) is in the range of 0.1 to 5 wt %, preferably 1 to 3 wt %, and the amount of component (C) is in the range of 0.1 to 5 wt %, preferably 1 to 3 wt %, based on the total amount of a diacetal as component (A), component (B) and component (C). If the amounts of both components (B) and (C) are less than 0.1 wt %, the effect of suppressing aldehyde generation is so weak that odor and taste evaluations show a tendency to be adversely affected. On the other hand, if the amounts of both components (B) and (C) are more than 5 wt %, nucleating agent characteristics as a clarifier of polyolefin resin tend to deteriorate.

The weight ratio of component (B) to component (C) is in the range of 1:0.2 to 5, preferably 1:0.5 to 3. Within this range, the combined use of components (B) and (C) tends to be remarkably effective.

Diacetal Composition of the Present Invention

The diacetal composition of the present invention comprises said amount of the agent for suppressing odor and taste, with the balance being diacetal (A) represented by the formula (1).

Specifically, the diacetal composition is a powdery or granular diacetal composition comprising:
i) diacetal (A) represented by the formula (1) and at least one member (component (B)) selected from the group consisting of (B1) saturated or unsaturated aliphatic alcohols and (B2) saturated or unsaturated aliphatic carboxylic acids having at least one hydroxyl group per molecule; or
ii) diacetal (A) represented by the formula (1), at least one member (component (B)) selected from the group consisting of (B1) saturated or unsaturated aliphatic alcohols and (B2) saturated or unsaturated aliphatic carboxylic acids having at least one hydroxyl group per molecule, and at least one member (component (C)) selected from the group consisting of (C1) anionic surfactants, (C2) alkali metal salts of fatty acids and (C3) aliphatic amines.

When the diacetal composition of the invention comprises components (A) and (B), it is preferable that the amount of component (B) is in the range of 0.1 to 10 wt %, preferably 1 to 5 wt % based on the diacetal composition, with the balance being diacetal (A) represented by the formula (1). If the amount of component (B) is less than 0.1 wt %, the effect of suppressing aldehyde generation is so weak that odor and taste evaluations show a tendency to be adversely affected. On the other hand, if component (B) is used in an amount of more than 10 wt %, it is rather difficult to give further effect of suppressing aldehyde generation, and nucleating agent characteristics as a clarifier of polyolefin resins tend to deteriorate.

When the diacetal composition according to the present invention is composed of components (A), (B) and (C), it is preferable that the amount of component (B) is in the range of 0.1 to 5 wt %, preferably 1 to 3 wt %, and the amount of component (C) is in the range of 0.1 to 5 wt %, preferably 1 to 3 wt %, based on the diacetal composition, with the balance being diacetal (A) represented by the formula (1). If the amounts of both components (B) and (C) are less than 0.1 wt %, the effect of suppressing aldehyde generation is so weak that odor and taste evaluations show a tendency to be adversely affected. On the other hand, if the amounts of both components (B) and (C) are more than 5 wt %, nucleating agent characteristics as a clarifier of polyolefin resins tend to deteriorate.

The weight ratio of component (B) to component (C) is in the range of 1:0.2 to 5, preferably 1:0.5 to 3. Within this range, the combined use of components (B) and (C) tends to be remarkably effective.

The diacetal composition according to the present invention is not particularly limited and suitably selected. Typical preferred examples are a combination of the following components (A) and (B), or a combination of the following components (A), (B) and (C):
component (A): at least one member selected from the group consisting of 1,3:2,4-O-dibenzylidene-D-sorbitol, 1,3:2,4-bis-O-(p-methylbenzylidene)-D-sorbitol, 1,3:2,4-bis-O-(p-ethylbenzylidene)-D-sorbitol, and 1,3:2,4-bis-O-(3,4-dimethylbenzylidene)-D-sorbitol,
component (B): at least one member selected from the group consisting of 9-hydroxystearic acid, 10-hydroxystearic acid, 12-hydroxystearic acid, 9,10-dihydroxystearic acid, lauryl alcohol, myristyl alcohol, palmityl alcohol, stearyl alcohol and behenyl alcohol,
component (C): at least one member selected from the group consisting of lithium lauryl sulfate, sodium lauryl sulfate, potassium lauryl sulfate, lithium polyoxyethylene (the number of moles of ethylene oxide added=2 to 3) lauryl sulfate, sodium polyoxyethylene (the number of moles of ethylene oxide added=2 to 3) lauryl sulfate, potassium polyoxyethylene (the number of moles of ethylene oxide added=2 to 3) lauryl sulfate, sodium laurate, potassium laurate, sodium myristate, potassium myristate, sodium palmitate, potassium palmitate, sodium stearate, potassium stearate, sodium behenate, sodium montanate, sodium 12-hydroxystearate, potassium 12-hydroxystearate, diethanolamine and diisopropanolamine.

Preferred examples of the diacetal composition comprising (A) and (B) include the following:
1,3:2,4-O-dibenzylidene-D-sorbitol+lauryl alcohol,
1,3:2,4-O-dibenzylidene-D-sorbitol+myristyl alcohol,
1,3:2,4-O-dibenzylidene-D-sorbitol+palmityl alcohol,
1,3:2,4-O-dibenzylidene-D-sorbitol+stearyl alcohol,
1,3:2,4-O-dibenzylidene-D-sorbitol+12-hydroxystearic acid,
1,3:2,4-bis-O-(p-methylbenzylidene)-D-sorbitol+lauryl alcohol,
1,3:2,4-bis-O-(p-methylbenzylidene)-D-sorbltol+myristyl alcohol,
1,3:2,4-bis-O-(p-methylbenzylidene)-D-sorbitol+palmityl alcohol,
1,3:2,4-bis-O-(p-methylbenzylidene)-D-sorbitol+stearyl alcohol,
1,3:2,4-bis-O-(p-methylbenzylidene)-D-sorbitol+12-hydroxystearic acid,
1,3:2,4-bis-O-(p-ethylbenzylidene)-D-sorbitol+lauryl alcohol,
1,3:2,4-bis-O-(p-ethylbenzylidene)-D-sorbitol+myristyl alcohol,
1,3:2,4-bis-O-(p-ethylbenzylidene)-D-sorbitol+palmityl alcohol,
1,3:2,4-bis-O-(p-ethylbenzylidene)-D-sorbitol+stearyl alcohol,
1,3:2,4-bis-O-(p-ethylbenzylidene)-D-sorbitol+12-hydroxystearic acid,
1,3:2,4-bis-O-(3,4-dimethylbenzylidene)-D-sorbitol+lauryl alcohol,
1,3:2,4-bis-O-(3,4-dimethylbenzylidene)-D-sorbitol+myristyl alcohol,
1,3:2,4-bis-O-(3,4-dimethylbenzylidene)-D-sorbitol+palmityl alcohol,
1,3:2,4-bis-O-(3,4-dimethylbenzylidene)-D-sorbitol+stearyl alcohol,
1,3:2,4-bis-O-(3,4-dimethylbenzylidene)-D-sorbitol+12-hydroxystearic acid and the like.

Preferred examples of the diacetal composition comprising components (A), (B) and (C) include the following:
(1) 1,3:2,4-O-dibenzylid ene-D-sorbitol+lauryl alcohol+sodium stearate,
1,3:2,4-O-dibenzylidene-D-sorbitol+myristyl alcohol+sodium stearate,
1,3:2,4-O-dibenzylidene-D-sorbitol+palmityl alcohol+sodium stearate,
1,3:2,4-O-dibenzylidene-D-sorbitol+stearyl alcohol+sodium stearate,
1,3:2,4-O-dibenzylidene-D-sorbitol+12-hydroxystearic acid+sodium laurate,
1,3:2,4-O-dibenzylidene-D-sorbitol+12-hydroxystearic acid+sodium palmitate,
1,3:2,4-O-dibenzylidene-D-sorbitol+12-hydroxysteario acid+lithium stearate,
1,3:2,4-O-dibenzylidene-D-sorbitol+12-hydroxystearic acid+potassium stearate,
1,3:2,4-O-dibenzylidene-D-sorbitol+12-hydroxystearic acid+sodium 12-hydroxystearate,
1,3:2,4-O-dibenzylidene-D-sorbitol+12-hydroxystearic acid+sodium stearate,
1,3:2,4-O-dibenzylidene-D-sorbitol+12-hydroxystearic acid+potassium 12-hydroxystearate,
1,3:2,4-O-dibenzylidene-D-sorbitol+12-hydroxystearic acid+sodium behenate,
1,3:2,4-O-dibenzylidene-D-sorbitol+12-hydroxystearic acid+sodium montanate,
1,3:2,4-O-dibenzylidene-D-sorbitol+12-hydroxystearic acid+sodium oleate,
1,3:2,4-O-dibenzylidene-D-sorbitol+12-hydroxystearic acid+lithium lauryl sulfate,
1,3:2,4-O-dibenzylidene-D-sorbitol+12-hydroxystearic acid+sodium lauryl sulfate,
1,3:2,4-O-dibenzylidene-D-sorbitol+12-hydroxystearic acid+potassium lauryl sulfate,
1,3:2,4-O-dibenzylidene-D-sorbitol+12-hydroxystearic acid+potassium oleyl sulfate,
1,3:2,4-O-dibenzylidene-D-sorbitol+12-hydroxystearic acid+sodium polyethyleneoxy (3 moles added) lauryl sulfate,
1,3:2,4-O-dibenzylidene-D-sorbitol+12-hydroxystearic acid+sodium glyceryl monolaurate sulfate,
1,3:2,4-O-dibenzylidene-D-sorbitol+12-hydroxystearic acid+diethanolamine,
1,3:2,4-O-dibenzylidene-D-sorbitol+12-hydroxystearic acid+triisopropanolamine.
(2) 1,3:2,4-bis-O-(p-methylbenzylidene)-D-sorbitol+lauryl alcohol+sodium stearate,
1,3:2,4-bis-O-(p-methylbenzylidene)-D-sorbitol+myristyl alcohol+sodium stearate,
1,3:2,4-bis-O-(p-methylbenzylidene)-D-sorbitol+palmityl alcohol+sodium stearate,
1,3:2,4-bis-O-(p-methylbenzylidene)-D-sorbitol+stearyl alcohol+sodium stearate,
1,3:2,4-bis-O-(p-methylbenzylidene)-D-sorbitol+12-hydroxystearic acid+sodium laurate,
1,3:2,4-bis-O-(p-methylbenzylidene)-D-sorbitol+12-hydroxystearic acid+sodium palpitate,
1,3:2,4-bis-O-(p-methylbenzylidene)-D-sorbitol+12-hydroxystearic acid+lithium stearate,
1,3:2,4-bis-O-(p-methylbenzylidene)-D-sorbitol+12-hydroxystearic acid+sodium stearate,
1,3:2,4-bis-O-(p-methylbenzylidene)-D-sorbitol+12-hydroxystearic acid+potassium 12-hydroxystearate,
1,3:2,4-bis-O-(p-methylbenzylidene)-D-sorbitol+12-hydroxystearic acid+sodium behenate,
1,3:2,4-bis-O-(p-methylbenzylidene)-D-sorbitol+12-hydroxystearic acid+sodium montanate,
1,3:2,4-bis-O-(p-methylbenzylidene)-D-sorbitol+12-hydroxystearic acid+sodium oleate,
1,3:2,4-bis-O-(p-methylbenzylidene)-D-sorbitol+12-hydroxystearic acid+lithium lauryl sulfate,
1,3:2,4-bis-O-(p-methylbenzylidene)-D-sorbitol+12-hydroxystearic acid+sodium lauryl sulfate,
1,3:2,4-bis-O-(p-methylbenzylidene)-D-sorbitol+12-hydroxystearic acid+potassium lauryl sulfate,
1,3:2,4-bis-O-(p-methylbenzylidene)-D-sorbitol+12-hydroxystearic acid+potassium oleyl sulfate,
1,3:2,4-bis-O-(p-methylbenzylidene)-D-sorbitol+12-hydroxystearic acid+potassium stearate,
1,3:2,4-bis-O-(p-methylbenzylidene)-D-sorbitol+12-hydroxystearic acid+sodium 12-hydroxystearate,
1,3:2,4-bis-O-(p-methylbenzylidene)-D-sorbitol+12-hydroxystearic acid+sodium polyethyleneoxy (3 moles added) lauryl sulfate,
1,3:2,4-bis-O-(p-methylbenzylidene)-D-sorbitol+12-hydroxystearic acid+sodium glyceryl monolaurate sulfate,
1,3:2,4-bis-O-(p-methylbenzylidene)-D-sorbitol+12-hydroxystearic acid+diethanolamine, and
1,3:2,4-bis-O-(p-methylbenzylidene)-D-sorbitol+12-hydroxystearic acid+triisopropanolamine,
(3) 1,3:2,4-bis-O-(p-ethylbenzylidene)-D-sorbitol+lauryl alcohol+sodium stearate,
1,3:2,4-bis-O-(p-ethylbenzylidene)-D-sorbitol+myristyl alcohol+sodium stearate,
1,3:2,4-bis-O-(p-ethylbenzylidene)-D-sorbitol+palmityl alcohol+sodium stearate,
1,3:2,4-bis-O-(p-ethylbenzylidene)-D-sorbitol+stearyl alcohol+sodium stearate,
1,3:2,4-bis-O-(p-ethylbenzylidene)-D-sorbitol+12-hydroxystearic acid+sodium laurate,
1,3:2,4-bis-O-(p-ethylbenzylidene)-D-sorbitol+12-hydroxystearic acid+sodium palmitate,
1,3:2,4-bis-O-(p-ethylbenzylidene)-D-sorbitol+12-hydroxystearic acid+lithium stearate,
1,3:2,4-bis-O-(p-ethylbenzylidene)-D-sorbitol+12-hydroxystearic acid+sodium stearate,
1,3:2,4-bis-O-(p-ethylbenzylidene)-D-sorbitol+12-hydroxystearic acid+potassium 12-hydroxystearate,
1,3:2,4-bis-O-(p-ethylbenzylidene)-D-sorbitol+12-hydroxystearic acid+sodium behenate,
1,3:2,4-bis-O-(p-ethylbenzylidene)-D-sorbitol+12-hydroxystearic acid+potassium stearate,
1,3:2,4-bis-O-(p-ethylbenzylidene)-D-sorbitol+12-hydroxystearic acid+sodium 12-hydroxystearate,
1,3:2,4-bis-O-(p-ethylbenzylidene)-D-sorbitol+12-hydroxystearic acid+sodium montanate,
1,3:2,4-bis-O-(p-ethylbenzylidene)-D-sorbitol+12-hydroxystearic acid+sodium oleate,
1,3:2,4-bis-O-(p-ethylbenzylidene)-D-sorbitol+12-hydroxystearic acid+lithium lauryl sulfate,
1,3:2,4-bis-O-(p-ethylbenzylidene)-D-sorbitol+12-hydroxystearic acid+sodium lauryl sulfate,
1,3:2,4-bis-O-(p-ethylbenzylidene)-D-sorbitol+12-hydroxystearic acid+potassium lauryl sulfate,
1,3:2,4-bis-O-(p-ethylbenzylidene)-D-sorbitol+12-hydroxystearic acid+potassium oleyl sulfate,
1,3:2,4-bis-O-(p-ethylbenzylidene)-D-sorbitol+12-hydroxystearic acid+sodium polyethyleneoxy (3 moles added) lauryl sulfate,
1,3:2,4-bis-O-(p-ethylbenzylidene)-D-sorbitol+12-hydroxystearic acid+sodium glyceryl monolaurate sulfate,
1,3:2,4-bis-O-(p-ethylbenzylidene)-D-sorbitol+12-hydroxystearic acid+diethanolamine,
1,3:2,4-bis-O-(p-ethylbenzylidene)-D-sorbitol+12-hydroxystearic acid+triisopropanolamine.
(4) 1,3:2,4-bis-O-(3,4-dimethylbenzylidene)-D-sorbitol+lauryl alcohol+sodium stearate,
1,3:2,4-bis-O-(3,4-dimethylbenzylidene)-D-sorbitol+myristyl alcohol+sodium stearate,
1,3:2,4-bis-O-(3,4-dimethylbenzylidene)-D-sorbitol+palmityl alcohol+sodium stearate,
1,3:2,4-bis-O-(3,4-dimethylbenzylidene)-D-sorbitol+stearyl alcohol+sodium stearate,
1,3:2,4-bis-O-(3,4-dimethylbenzylidene)-D-sorbitol+12-hydroxystearic acid+sodium laurate,
1,3:2,4-bis-O-(3,4-dimethylbenzylidene)-D-sorbitol+12-hydroxystearic acid+sodium palmitate,
1,3:2,4-bis-O-(3,4-dimethylbenzylidene)-D-sorbitol+12-hydroxystearic acid+lithium stearate,
1,3:2,4-bis-O-(3,4-dimethylbenzylidene)-D-sorbitol+12-hydroxystearic acid+sodium stearate,
1,3:2,4-bis-O-(3,4-dimethylbenzylidene)-D-sorbitol+12-hydroxystearic acid+potassium 12-hydroxystearate,
1,3:2,4-bis-O-(3,4-dimethylbenzylidene)-D-sorbitol+12-hydroxystearic acid+potassium stearate,
1,3:2,4-bis-O-(3,4-dimethylbenzylidene)-D-sorbitol+12-hydroxystearic acid+sodium 12-hydroxystearate,
1,3:2,4-bis-O-(3,4-dimethylbenzylidene)-D-sorbitol+12-hydroxystearic acid+sodium behenate,
1,3:2,4-bis-O-(3,4-dimethylbenzylidene)-D-sorbitol+12-hydroxystearic acid+sodium montanate,
1,3:2,4-bis-O-(3,4-dimethylbenzylidene)-D-sorbitol+12-hydroxystearic acid+sodium oleate,
1,3:2,4-bis-O-(3,4-dimethylbenzylidene)-D-sorbitol+12-hydroxystearic acid+lithium lauryl sulfate,
1,3:2,4-bis-O-(3,4-dimethylbenzylidene)-D-sorbitol+12-hydroxystearic acid+sodium lauryl sulfate,
1,3:2,4-bis-O-(3,4-dimethylbenzylidene)-D-sorbitol+12-hydroxystearic acid+potassium lauryl sulfate,
1,3:2,4-bis-O-(3,4-dimethylbenzylidene)-D-sorbitol+12-hydroxystearic acid+potassium oleyl sulfate,
1,3:2,4-bis-O-(3,4-dimethylbenzylidene)-D-sorbitol+12-hydroxystearic acid+sodium polyethyleneoxy (3 moles added) lauryl sulfate,
1,3:2,4-bis-O-(3,4-dimethylbenzylidene)-D-sorbitol+12-hydroxystearic acid+sodium glyceryl monolaurate sulfate,
1,3:2,4-bis-O-(3,4-dimethylbenzylidene)-D-sorbitol+12-hydroxystearic acid+diethanolamine,
1,3:2,4-bis-O-(3,4-dimethylbenzylidene)-D-sorbitol+12-hydroxystearic acid+triisopropanolamine, and the like.

The diacetal composition according to the present invention can be easily prepared by adding component (B) or components (B) and (C) to the diacetals represented by the formula (A).

The method of adding the components is not particularly limited insofar as a desired diacetal composition can be obtained. Examples of such method include: i) addition in the course of manufacturing a diacetal represented by the formula (1) from sorbitol and a corresponding benzaldehyde according to various known processes (such as those set forth in Japanese Unexamined Patent Publication No. 1990-231488); ii) powder mixing with a diacetal represented by the formula (1) by means of a mixer such as Henschel mixer, V-blender, ribbon blender or the like; and iii) addition and mixing of the above specified amount of component (B) or components (B) and (C) as such or as dissolved in a solvent such as methanol, ethanol and like alcohols having about 1-3 carbon atoms, water and the like to a slurry of a diacetal represented by the formula (1) in a dispersion medium such as methanol, ethanol and like alcohols having about 1-3 carbon atoms, water and the like, and subsequent removal of the solvent by evaporation.

In the above process i), it is preferable that the specified amount of component (B) or components (B) and (C) is added after a diacetal represented by the formula (1) is formed by diacetalization reaction of a sorbitol and a benzaldehyde.

In the above process iii), the diacetal concentration in the slurry is not particularly limited and in general it is preferably about 10 to 60 wt %. The slurry temperature is suitably selected from a wide range and in general it is preferably 20 to 100° C.

The thus obtained diacetal composition containing component (B) or components (B) and (C) is then pulverized, crushed, granulated or classified, if necessary.

The diacetal composition of the present invention may take any form as suitably selected, and may be in the common form of powders or grains, or in a granulated form such as granules, cylinders, pellets and the like.

In the case of powders, the average particle diameter thereof is 3 to 2000 μm and is preferably 7 to 200 μm. If the average particle diameter is smaller than 3 μm, powder characteristics tends to be poor, and a special pulverization apparatus is necessary.

In the case of granules, a granular diacetal composition of the desired shape and size can be obtained from a powdery diacetal composition obtained by the above method. This granular diacetal composition is advantageous in that it has reduced dust generation and improved particle fluidity, as compared with a powdery diacetal composition.

Any of the above forms can be manufactured using a known granulator, pulverizer/crusher, classifier, or the like. Examples of granulators include dry or wet extrusion granulators, mixing and stirring granulators, tableting machines, dry compression roll granulators, and oscillating granulator. Examples of pulverizer/crushers include pin mills, Jet mills, pulverizers, cutter mills, hammer mills, planar crushers, flake crushers, nibblers, etc. Examples of classifiers include vibrating sifters, air classifiers, etc.

In the diacetal composition thus obtained, transfer of odor and taste originating from the diacetal represented by the formula (1) is suppressed.

Accordingly, the present invention provides a method for suppressing transfer of odor and taste originating from the diacetal or a method for suppressing aldehyde generation by thermal decomposition of the diacetal, comprising adding component (B) singly or adding a combination of components (B) and (C) to the diacetal represented by the formula (1).

Furthermore, the present invention also provides use of component (B) or a combination of components (B) and (C) for suppressing transfer of odor and taste originating from the diacetal or for suppressing aldehyde generation by thermal decomposition of the diacetal.

Polyolefin Resin Nucleating Agent

The polyolefin resin nucleating agent of the present invention may be the present diacetal composition itself or may be prepared by adding a polyolefin resin additive to the diacetal composition.

The diacetal composition used for the present polyolefin resin nucleating agent is useful since it shows extremely low aldehyde generation due to thermal hysteresis and very little decrease in nucleating agent performances. If the present nucleating agent includes an antioxidant, its storage stability can be remarkably improved. Such nucleating agent having good storage stability comprises an antioxidant in an amount of 0.01 to 5 weight parts, preferably 0.01 to 3 weight parts, per 100 weight parts of the diacetal composition.

Examples of such antioxidants include phenol-containing antioxidants, phosphite-containing compounds, sulfur-containing antioxidants and the like. More specifically, 2,6-di-tert-butylphenol, n-octadecyl-3-(3′,5′-di-tert-butyl-4′-hydroxyphenyl)propionate, tetrakis[methylene-3-(3,5-di-tert-butyl-4-hydroxyphenyl)propionate]methane, tris(3,5-di-tert-butyl-4-hydroxybenzyl)isocyanurate, 4,4′-butylidene-bis(3-methyl-6-tert-butylphenol), triethyleneglycol-bis-[3-(3-tert-butyl-4-hydroxy-5-methylphenyl)propionate] and the like are recommended. Among them, tetrakis[methylene-3-(3,5-di-tert-butyl-4-hydroxyphenyl)propionate]methane is especially recommended.

According to purpose or application, a conventional polyolefin modifier can be suitably added to the polyolefin resin nucleating agent of the present invention, insofar as the effect of the present invention is not compromised.

Examples of such polyolefin modifiers include the various additives listed in “The Tables of Positive Lists of Additives” edited by the Japan Hygienic Olefin and Styrene Plastic Association (January, 2002). More specific examples include stabilizers (such as metal compounds, epoxy compounds, nitrogen compounds, phosphorus compounds, and sulfur compounds), UV absorbers (such as benzophenone compounds and benzotriazole compounds), surfactants, lubricants (such as paraffin, wax, and other aliphatic hydrocarbons, C₈ to C₂₂ higher fatty acids, C₈ to C₂₂ higher fatty acid metal (Al, Ca, Mg, Zn) salts, C₈ to C₂₂ higher aliphatic alcohols, polyglycols, esters of C₄ to C₂₂ higher fatty acids and C₄ to C₁₈ aliphatic monohydric alcohols, C₈ to C₂₂ higher fatty acid amides, silicone oils, and rosin derivatives), fillers (such as talc, hydrotalcite, mica, zeolite, perlite, diatomaceous earth, calcium carbonate, and glass fibers), foaming agents, foaming auxiliaries, polymer additives, plasticizers (such as dialkyl phthalates and dialkyl hexahydrophthalates), crosslinking agents, crosslinking accelerators, antistatic agents, flame retardants, dispersants, organic and inorganic pigments, working auxiliaries, other nucleating agents, and like additives.

Various methods can be employed to mix these additional components. For example, it is preferable to dry blend the diacetal composition of the present invention with the additional components to obtain a uniform mixture.

Polyolefin Resin Composition

The polyolefin resin composition according to the present invention is obtained by incorporating the polyolefin resin nucleating agent of the present invention into a polyolefin resin by a standard method.

The polyolefin resin composition of the present invention can be manufactured by any conventional method, with no particular restrictions thereon, as long as the desired resin composition is obtained. For example, a polyolefin resin (powder or flakes), the polyolefin resin nucleating agent of the present invention, and, if needed, a polyolefin modifier discussed below are mixed in a conventional mixer, such as Henschel mixer, V-blender or ribbon blender, to obtain a blend type of polyolefin resin composition. Other examples include a method in which this blend type of polyolefin resin composition is melt kneaded at the desired temperature in a conventional kneader, such as a single screw or twin screw extruder, the extruded strands are cooled, and the strands thus obtained are cut into pellets, as well as a method that is a variation of this pellet type, in which master batch pellets are made from a polyolefin resin nucleating agent and a polyolefin resin.

There are no particular restrictions on the amount of the polyolefin resin nucleating agent according to the present invention to be added to a polyolefin resin, as long as the desired effect is obtained, and this amount can be suitably selected from a wide range. Usually, the amount is 0.05 to 3 weight parts, preferably 0.07 to 1 weight parts, per 100 weight parts of the polyolefin resin. Blending within this range can fully produce the desired effect of the present invention.

A method for adding the diacetal composition to a polyolefin resin may preferably be a direct addition using a conventional machine such as a single screw or twin screw extruder, but is not limited thereto. Alternatively, addition in the form of high concentration master batch of, for example, about 2 to 15 wt % is also allowable.

Examples of the polyolefin resin according to the present invention include polyethylene-based resins, polypropylene-based resins, polybutene-based resins, polymethylpentene-based resins, and polybutadiene-based resins. Specific examples are high-density polyethylene, medium-density polyethylene, linear polyethylene, ethylene copolymers with an ethylene content of at least 50 wt % and preferably 70 wt % or higher, propylene homopolymers, propylene copolymers with a propylene content of at least 50 wt % and preferably 70 wt % or higher, butene homopolymers, butene copolymers with a butene content of at least 50 wt % and preferably 70 wt % or higher, methylpentene homopolymers, methylpentene copolymers with a methylpentene content of at least 50 wt % and preferably 70 wt % or higher, and polybutadiene.

The above copolymers may be random copolymers or block copolymers. The stereoregularity of these resins may be either isotactic or syndiotactic.

Specific examples of comonomers which can form the various copolymers above include ethylene, propylene, butene, pentene, hexene, heptene, octene, nonene, decene, undecene, dodecene and like C₂ to C₁₂ α-olefins; 1,4-endomethylenecyclohexene and like bicyclo-type monomers; methyl (meth)acrylate, ethyl (meth)acrylate, and like (meth)acrylic esters; vinyl acetate; and the like.

Catalysts that can be used in the manufacture of these polymers include not only Ziegler-Natta catalysts which are commonly used, but also a catalyst system comprising a combination of a transition metal compound (e.g., a titanium halide such as titanium trichloride or titanium tetrachloride) supported on a support comprising as a main component magnesium chloride or like magnesium halide, with an alkyl aluminum compound (such as triethyl aluminum or diethyl aluminum chloride), and metallocene catalysts.

The recommended melt flow rate (hereinafter referred to as “MFR”, measured according to JIS K 7210-1976) of the polyolefin resin according to the present invention can be suitably selected according to the molding method to be employed, and is usually 0.01 to 200 g/10 minutes, preferably 0.05 to 100 g/10 minutes.

To the extent that the effect of the present invention is not lost, the above-mentioned known polyolefin modifiers can be added to the resin composition of the present invention as dictated by the intended use and application thereof.

Thus obtained polyolefin resin composition according to the present invention is novel and useful having superior nucleating agent performances such as clarity and the like, in which aldehyde generation during the molding process as well as transfer of aldehyde odor and taste in the final molded product are suppressed.

Accordingly, in a polyolefin resin composition comprising a diacetal represented by the formula (1), the present invention also provides a method for suppressing odor originating from the diacetal or a method for suppressing aldehyde generation by thermal decomposition of the diacetal, which comprises adding the polyolefin resin nucleating agent of the invention to a polyolefin resin.

In addition, in a polyolefin resin composition comprising a diacetal represented by the formula (1), the present invention also provides use of the polyolefin resin nucleating agent of the invention for suppressing transfer of odor and taste originating from the diacetal or for suppressing aldehyde generation by thermal decomposition of the diacetal.

Polyolefin Resin Moldings

The polyolefin resin moldings of the present invention are obtained by molding the polyolefin resin composition of the present invention according to a conventional molding method. Any known method, such as injection molding, extrusion molding, blow molding, pressure forming, rotational molding, or film forming, can be employed in molding the polyolefin resin composition according to the present invention. The processing conditions can be suitably selected from the wide range of conditions that have been employed in the past.

The polyolefin resin composition of the present invention can be put into use and then molded in the same fields in which polyolefin resin compositions containing a DBS or the like as a nucleating agent have been conventionally used.

In the polyolefin resin molded product according to the present invention, a generation of aldehyde and the like during molding process is suppressed and therefore odor generation is decreased, compared with a molded product prepared from the conventional resin composition containing DBS or the like.

Accordingly, the present invention provides a method for suppressing odor due to aldehyde derived from diacetal at the time of molding a polyolefin resin, comprising mixing the present nucleating agent with the polyolefin resin and molding a resultant resin composition.

In addition, since the amount of aldehyde and the like is significantly reduced in the molded product of the invention, transfer of odor and taste due to aldehyde and the like is also decreased. Furthermore, the polyolefin resin molded product of the present invention shows superior clarity.

The present invention provides a method for suppressing transfer of odor and taste originating from a diacetal to foods, cosmetics, medicines and like content, characterized in that said method comprises placing the content in a packaging material or a container prepared by mixing the nucleating agent of the present invention with a polyolefin resin and molding the resultant resin composition.

Thus the polyolefin resin molded product of the invention can be used advantageously particularly in the fields of packaging materials for foods, containers for foods, cosmetics, medicines and the like. Of course, it is also used in other fields. Examples include medical instruments sterilized by heat, radiation or the like, such as disposable syringes, infusion and transfusion sets, equipment for collecting blood, etc.; packaging materials for foods, plants, etc. sterilized by radiation or the like; cases such as cases for clothes, containers for clothes, etc.; cups for heat-packaging foods, packaging containers for retort-processed foods; containers for use in microwave oven, containers for cans, vessels, etc. for beverages such as juice, tea, etc., cosmetics, medicines, shampoo, etc.; containers and caps for seasonings such as miso, soy sauce, etc.; cases and containers for foods such as water, rice, bread, pickles, etc.; sundries such as cases for use in refrigerators, etc.; stationery; electric and mechanical parts; automobile parts, etc.

### EXAMPLES

The present invention will now be described in detail with reference to examples and comparative examples, but the present invention is not limited to or by these examples.

### Examples 1-5

1,3:2,4-di(p-methylbenzylidene)sorbitol (hereinafter referred to as “Me-DBS”) and an alcohol or 12-hydroxystaric acid shown in Table 1 were stirred for one hour in methanol (weighing as 6 times as Me-DBS) under reflux to give a mixture in a white paste form. Then methanol was removed under reduced pressure, dried in a vacuum drier for one hour under the pressure of 133 Pa at a temperature of 80° C., to obtain a powdery sample (hereinafter referred to as “Me-DBS composition”).

To 100 weight parts of an isotactic random polypropylene resin with an ethylene content of 3.0 wt % (MFR=20 g/10 minutes; hereinafter referred to as “r-PP”), were added 0.2 weight part of the Me-DBS composition, 0.05 weight part of tetrakis[methylene-3-(3,5-di-tert-butyl-4-hydroxyphenyl)propionate]methane (trade name “Irganox 1010”, made by Ciba Specialty Chemicals) and 0.05 weight part of calcium stearate, and these components were blended in a Henschel mixer. Then the mixture was melt kneaded using a single screw extruder having a diameter of 25 mm at a resin temperature of 240° C. and pelletized. The odor evaluation of the obtained pellets was carried out by the following dry method. The results are shown in Table 1.

Odor Evaluation of Pellets by Dry Method

A 225 ml glass bottle in which 60 g of pellets were sealed was left to stand in a constant temperature bath of 80° C. for 2 hours, and cooled to room temperature. Immediately after that, the odor intensity was rated by ten panelists. The rating criteria used by the panelists were as follows: zero point, no unpleasant odor; 1 point, slightly unpleasant odor; 2 point, distinct unpleasant odor; 3 point, strong unpleasant odor. Total value of the points determined by the ten panelists was used for evaluation.

The injection molding of the obtained pellet was carried out at a resin temperature of 260° C. and a mold temperature of 40° C. to give a test piece. Odor evaluation by dry and wet methods, measurement of aldehyde generation and taste evaluation was performed by the following methods using the obtained injection-molded product. The result is shown in Table 1. In addition, measurement of crystallization temperature (Tc) and a haze value (%) was performed for evaluating the nucleating agent performance.

Method of Measuring Crystallization Temperature (Tc)

According to JIS K7121, the crystallization temperature was measured using a differential scanning calorimeter (trade name “DSC7” from Perkin Elmer). The higher the Tc value, the quicker the crystallization rate is, and the molding cycle can be shortened.

Haze Value (Improvement in Clarity)

The haze value was measured using a haze meter from Toyo Seki Seisakusho according to JIS K 6714 and JIS K 6717. The smaller the measured value, the better the clarity is.

Subsequently, the obtained injection-molded product was subjected to the odor evaluation by the following dry and wet methods. The results are shown in Table 1.

Odor Evaluation of Injection-Molded Product by Dry Method

A 225 ml glass bottle in which 20 g of test piece were sealed was left to stand in a constant temperature bath of 80° C. for 2 hours, and cooled to room temperature. Immediately after that, the odor intensity was rated by ten panelists. The rating criteria used by the panelists were as follows: zero point, no unpleasant odor; 1 point, slightly unpleasant odor; 2 points, distinct unpleasant odor; 3 points, strong unpleasant odor. Total value of the points determined by the ten panelists was used for evaluation.

After sealing 20 g of test piece and 140 g of deionized water in a 225 ml glass bottle, the bottle was left in a constant temperature bath of 80° C. for 2 hours and cooled to room temperature. Odor evaluations, measurement of aldehyde generation and taste evaluation was performed by the following methods using the obtained solution as an evaluation sample.

Odor Evaluation of Injection-Molded Product by Wet Method

The evaluation sample was subjected to the odor intensity rating by ten panelists. The rating criteria used by the panelists were as follows: zero point, no unpleasant odor; 1 point, slightly unpleasant odor; 2 points, distinct unpleasant odor; 3 points, strong unpleasant odor. Total value of the points determined by the ten panelists was used for evaluation.

Aldehyde Generation in Injection-Molded Product

Aldehyde content of the evaluation sample was measured using a high performance liquid chromatography. The aldehyde amount was expressed in μg/PPg, I.e. μg per 1 g of test piece.

Taste Evaluation of Injection-Molded Product

Ten gram of the evaluation sample was placed in a cup made of glass and subjected to the taste evaluation by ten panelists. In the taste evaluation, ten panelists evaluated the evaluation sample in comparison with control samples prepared by the following method, wherein the rating criteria used by the panelists were as follows: zero point, no difference; 1 point, slight difference; 2 points, distinct difference. Total value of the points determined by the ten panelists was used for evaluation.

Preparation of Control Samples for Taste Evaluation

The solutions as control samples for taste evaluation were prepared in the same manner as in Example 1 except that DBSs were not added.

In the diacetal compositions shown in Table 1, the amount of component (B) is expressed as a percentage (% by weight) based on the total amount of components (A) and (B). Accordingly, the percentage of component (A), MA (wt %), can be calculated as follows: MA=100-MB, wherein MB is the percentage of component (B) (wt %).

TABLE 1

| | | | | | | | | | Odor evaluation | | | | |
|---|---|---|---|---|---|---|---|---|---|---|---|---|---|
| | | Diacetal composition | | | | | | | | Injection-molded | | | |
| | | Com- | | | | | Crystallization | Haze | Pellet | product | | Aldehyde | Taste |
| | | ponent | Component | Amount | Component | Amount | temp. | value | Dry | Dry | Wet | generation | eval- |
| Ex. | Resin | (A) | (B) | (wt. %) | (C) | (wt. %) | (° C.) | (%) | method | method | method | (μg/PPg) | uation |
| 1 | r-PP | Me-DBS | lauryl alcohol | 2.5 | — | — | 127 | 12 | 8 | 10 | 9 | 4.0 | 10 |
| 2 | r-PP | Me-DBS | myristyl alcohol | 2.5 | — | — | 128 | 13 | 8 | 9 | 9 | 4.2 | 10 |
| 3 | r-PP | Me-DBS | palmityl alcohol | 2.5 | — | — | 128 | 12 | 8 | 9 | 9 | 4.1 | 10 |
| 4 | r-PP | Me-DBS | stearyl alcohol | 2.5 | — | — | 127 | 13 | 8 | 10 | 9 | 4.0 | 10 |
| 5 | r-PP | Me-DBS | 12-hydroxystearic | 2.5 | — | — | 128 | 11 | 7 | 9 | 8 | 3.7 | 9 |
| | | | acid | | | | | | | | | | |

### Examples 6-27

Evaluation was conducted in the same manner as in Examples 1-5, except that fatty acid alkali metal salts, sulfuric ester salts and amines were employed in the amounts shown in Tables 2 and 3 in addition to the alcohols or 12-hydroxystearic acid shown in Tables 2 and 3. The results are shown in Tables 2 and 3.

In the diacetal compositions shown in Tables 2 and 3, the amount of component (B) and component (C) are expressed as a percentage (% by weight) based on the total amount of components (A) and (B). Accordingly, the percentage of component (A), MA (wt %), can be calculated as follows: MA=100−(MB+MC), wherein MB is the percentage of component (B) (wt %) and MC is the percentage of component (C) (wt %), with the proviso that MC=0, if component (C) was not used. The same applies to the following Tables 4 and 5.

TABLE 2

| | | | | | | | | | Odor evaluation | | | | |
|---|---|---|---|---|---|---|---|---|---|---|---|---|---|
| | | Diacetal composition | | | | | Crys- | | | Injection-molded | | | |
| | | Com- | | | | | tallization | Haze | Pellet | product | | Aldehyde | Taste |
| | | ponent | | Amount | Component | Amount | temp. | value | Dry | Dry | Wet | generation | evalu- |
| Ex. | Resin | (A) | Component (B) | (wt. %) | (C) | (wt. %) | (° C.) | (%) | method | method | method | (μg/PPg) | ation |
| 6 | r-PP | Me- | lauryl alcohol | 2.5 | Na stearate | 2.5 | 127 | 12 | 5 | 5 | 5 | 3.1 | 5 |
| | | DBS | | | | | | | | | | | |
| 7 | r-PP | Me- | myristyl alcohol | 2.5 | Na stearate | 2.5 | 128 | 12 | 4 | 5 | 4 | 3.0 | 5 |
| | | DBS | | | | | | | | | | | |
| 8 | r-PP | Me- | palmityl alcohol | 2.5 | Na stearate | 2.5 | 127 | 13 | 6 | 5 | 5 | 2.9 | 5 |
| | | DBS | | | | | | | | | | | |
| 9 | r-PP | Me- | stearyl alcohol | 2.5 | Na stearate | 2.5 | 127 | 12 | 5 | 4 | 5 | 3.0 | 5 |
| | | DBS | | | | | | | | | | | |
| 10 | r-PP | Me- | 12-hydroxystearic | 2.5 | Na laurate | 2.5 | 128 | 12 | 4 | 5 | 5 | 2.9 | 5 |
| | | DBS | acid | | | | | | | | | | |
| 11 | r-PP | Me- | 12-hydroxystearic | 2.5 | Na palmitate | 2.5 | 127 | 12 | 4 | 4 | 5 | 3.0 | 5 |
| | | DBS | acid | | | | | | | | | | |
| 12 | r-PP | Me- | 12-hydroxystearic | 2.5 | Li stearate | 2.5 | 128 | 13 | 5 | 4 | 4 | 3.0 | 5 |
| | | DBS | acid | | | | | | | | | | |
| 13 | r-PP | Me- | 12-hydroxystearic | 2.5 | Na stearate | 2.5 | 128 | 12 | 4 | 4 | 4 | 3.0 | 5 |
| | | DBS | acid | | | | | | | | | | |
| 14 | r-PP | Me- | 12-hydroxystearic | 2.5 | K stearate | 2.5 | 128 | 13 | 5 | 4 | 4 | 2.9 | 5 |
| | | DBS | acid | | | | | | | | | | |
| 15 | r-PP | Me- | 12-hydroxystearic | 2.5 | Na | 2.5 | 127 | 12 | 3 | 4 | 3 | 2.6 | 4 |
| | | DBS | acid | | 12-hydroxystearate | | | | | | | | |
| 16 | r-PP | Me- | 12-hydroxystearic | 2.5 | Na behenate | 2.5 | 128 | 13 | 4 | 4 | 4 | 2.9 | 4 |
| | | DBS | acid | | | | | | | | | | |

TABLE 3

| | | | | | | | | | Odor evaluation | | | | |
|---|---|---|---|---|---|---|---|---|---|---|---|---|---|
| | | Diacetal composition | | | | | Crys- | | | Injection-molded | | | |
| | | Com- | | | | | tallization | Haze | Pellet | product | | Aldehyde | Taste |
| | | ponent | Component | Amount | Component | Amount | temp. | value | Dry | Dry | Wet | generation | evalu- |
| Ex. | Resin | (A) | (B) | (wt. %) | (C) | (wt. %) | (° C.) | (%) | method | method | method | (μg/PPg) | ation |
| 17 | r-PP | Me- | 12-hydroxystearic | 2.5 | Na montanate | 2.5 | 128 | 12 | 4 | 4 | 3 | 2.8 | 4 |
| | | DBS | acid | | | | | | | | | | |
| 18 | r-PP | Me- | 12-hydroxystearic | 2.5 | Na oleate | 2.5 | 127 | 13 | 4 | 3 | 4 | 2.8 | 4 |
| | | DBS | acid | | | | | | | | | | |
| 19 | r-PP | Me- | 12-hydroxystearic | 2.5 | Na lauryl sulfate | 2.5 | 127 | 12 | 4 | 3 | 4 | 2.7 | 4 |
| | | DBS | acid | | | | | | | | | | |
| 20 | r-PP | Me- | 12-hydroxystearic | 2.5 | K lauryl sulfate | 2.5 | 127 | 13 | 4 | 4 | 3 | 2.8 | 4 |
| | | DBS | acid | | | | | | | | | | |
| 21 | r-PP | Me- | 12-hydroxystearic | 2.5 | Li lauryl sulfate | 2.5 | 128 | 13 | 3 | 4 | 3 | 2.9 | 5 |
| | | DBS | acid | | | | | | | | | | |
| 22 | r-PP | Me- | 12-hydroxystearic | 2.5 | Ammonium | 2.5 | 127 | 12 | 5 | 4 | 4 | 2.7 | 4 |
| | | DBS | acid | | lauryl sulfate | | | | | | | | |
| 23 | r-PP | Me- | 12-hydroxystearic | 2.5 | K oleyl sulfate | 2.5 | 128 | 12 | 4 | 4 | 4 | 2.8 | 4 |
| | | DBS | acid | | | | | | | | | | |
| 24 | r-PP | Me- | 12-hydroxystearic | 2.5 | Na polyoxyethylene | 2.5 | 127 | 13 | 3 | 3 | 4 | 3.0 | 5 |
| | | DBS | acid | | (3 moles added) | | | | | | | | |
| | | | | | lauryl ether sulfate | | | | | | | | |
| 25 | r-PP | Me- | 12-hydroxystearic | 2.5 | Na glyceryl | 2.5 | 127 | 12 | 4 | 4 | 3 | 3.1 | 5 |
| | | DBS | acid | | monolaurate sulfate | | | | | | | | |
| 26 | r-PP | Me- | 12-hydroxystearic | 2.5 | Diethanolamine | 2.5 | 128 | 13 | 5 | 4 | 4 | 3.3 | 5 |
| | | DBS | acid | | | | | | | | | | |
| 27 | r-PP | Me- | 12-hydroxystearic | 2.5 | Triisopropanolamine | 2.5 | 127 | 13 | 5 | 5 | 4 | 3.2 | 5 |
| | | DBS | acid | | | | | | | | | | |

### Comparative Example 1

Evaluation was conducted in the same manner as in Example 1, except that Me-DBS not containing lauryl alcohol was used. The results are shown in Table 6.

### Examples 28-30

Evaluation was conducted in the same manner as in Examples 5, 13 and 19, except that 1,3:2,4-dibenzylidene sorbitol (hereinafter referred to as “DBS”) was used instead of Me-DBS. The results are shown in Table 4.

### Comparative Example 2

Evaluation was conducted in the same manner as in Example 28, except that DBS not containing 12-hydroxystearic acid was used. The results are shown in Table 6.

### Examples 31-33

Evaluation was conducted in the same manner as in Examples 5, 13 and 19, except that 1,3:2,4-di(p-ethylbenzylidene)sorbitol (hereinafter referred to as “Et-DBS”) was used instead of Me-DBS. The results are shown in Table 4.

### Comparative Example 3

Evaluation was conducted in the same manner as in Example 31, except that Et-DBS not containing 12-hydroxystearic acid was used. The results are shown in Table 6.

### Examples 34-36

Evaluation was conducted in the same manner as in Examples 5, 13 and 19, except that 1,3:2,4-di(3,4-dimethylbenzylidene)sorbitol (hereinafter referred to as “3,4-DMDBS”) was used instead of Me-DBS. The results are shown in Table 4.

TABLE 4

| | | | | | | | | | Odor evaluation | | | | |
|---|---|---|---|---|---|---|---|---|---|---|---|---|---|
| | | | | | | | Crys- | | | Injection-molded | | | |
| | | Diacetal composition | | | | | tallization | Haze | Pellet | product | | Aldehyde | Taste |
| | | Component | Component | Amount | | Amount | temp. | value | Dry | Dry | Wet | generation | evalu- |
| Ex. | Resin | (A) | (B) | (wt. %) | Component (C) | (wt. %) | (° C.) | (%) | method | method | method | (μg/PPg) | ation |
| 28 | r-PP | DBS | 12-hydroxystearic | 2.5 | — | — | 118 | 22 | 7 | 8 | 9 | 4.1 | 7 |
| | | | acid | | | | | | | | | | |
| 29 | r-PP | DBS | 12-hydroxystearic | 2.5 | Na stearate | 2.5 | 117 | 21 | 6 | 5 | 5 | 3.2 | 4 |
| | | | acid | | | | | | | | | | |
| 30 | r-PP | DBS | 12-hydroxystearic | 2.5 | Na lauryl | 2.5 | 118 | 22 | 5 | 4 | 5 | 3.1 | 4 |
| | | | acid | | sulfate | | | | | | | | |
| 31 | r-PP | Et-DBS | 12-hydroxystearic | 2.5 | — | — | 126 | 19 | 6 | 8 | 7 | 3.9 | 6 |
| | | | acid | | | | | | | | | | |
| 32 | r-PP | Et-DBS | 12-hydroxystearic | 2.5 | Na stearate | 2.5 | 125 | 19 | 5 | 5 | 4 | 3.0 | 4 |
| | | | acid | | | | | | | | | | |
| 33 | r-PP | Et-DBS | 12-hydroxystearic | 2.5 | Na lauryl | 2.5 | 125 | 18 | 5 | 4 | 4 | 2.9 | 4 |
| | | | acid | | sulfate | | | | | | | | |
| 34 | r-PP | 3,4-DMDBS | 12-hydroxystearic | 2.5 | — | — | 127 | 13 | 6 | 7 | 7 | 3.5 | 5 |
| | | | acid | | | | | | | | | | |
| 35 | r-PP | 3,4-DMDBS | 12-hydroxystearic | 2.5 | Na stearate | 2.5 | 127 | 13 | 4 | 4 | 5 | 2.4 | 2 |
| | | | acid | | | | | | | | | | |
| 36 | r-PP | 3,4-DMDBS | 12-hydroxystearic | 2.5 | Na lauryl | 2.5 | 128 | 12 | 4 | 4 | 4 | 2.4 | 2 |
| | | | acid | | sulfate | | | | | | | | |

### Comparative Example 4

Evaluation was conducted in the same manner as in Example 34, except that 3,4-DMDBS not containing 12-hydroxystearic acid was used. The results are shown in Table 6.

### Comparative Example 5

Evaluation was conducted in the same manner as in Example 1, except that no nucleating agent was added. The results are shown in Table 6.

### Examples 37 and 38

Evaluation was conducted in the same manner as in Examples 5 and 19, except that an isotactic homopolypropylene resin (MFR=30 g/10 minutes, hereinafter referred to as “h-PP”) was used as a resin. The results are shown in Table 5.

### Comparative Example 6

Evaluation was conducted in the same manner as in Example 37, except that Me-DBS not containing 12-hydroxystearic acid was used. The results are shown in Table 6.

### Comparative Example 7

Evaluation was conducted in the same manner as in Example 37, except that no nucleating agent was added. The results are shown in Table 6.

### Examples 39 and 40

The dibenzylidene sorbitol nucleating agent of the present invention was prepared in the same manner as in Example 1, except that 12-hydroxystearic acid, sodium lauryl sulfate and Me-DBS were used in the amounts shown in Table 5.

To 100 weight parts of a linear low density polyethylene resin (density=0.926 g/cm3, MFR=20 g/10 minutes, hereinafter referred to as “LLDPE”), 0.2 weight part of the dibenzylidene sorbitol nucleating agent was added, and these components were blended in a Henschel mixer. Then the mixture was melt kneaded using a single screw extruder having a diameter of 25 mm at a temperature of 200° C. and pelletized. The odor evaluation of the obtained pellets was carried out by the following dry method. The results are shown in Table 5.

Odor Evaluation of Pellets by Dry Method

A 225 ml glass bottle in which 60 g of the pellets were sealed was left to stand in a constant temperature bath of 40° C. for 2 hours, and cooled to room temperature. Immediately after that, the odor intensity was rated by ten panelists. The rating criteria used by the panelists were as follows: zero point, no unpleasant odor; 1 point, slightly unpleasant odor; 2 point, distinct unpleasant odor; 3 point, strong unpleasant odor. Total value of the points determined by the ten panelists was used for evaluation.

The injection molding of the obtained pellets was carried out at a resin temperature of 220° C. and a mold temperature of 30° C. to give a test piece. Crystallization temperature (To) and haze value (%) of the resultant injection-molded product were measured by the methods employed in Example 1. The results are shown in Table 5.

Subsequently, the obtained injection-molded product was subjected to the odor evaluation by the following dry method. The results are shown in Table 5.

Odor Evaluation of Injection-Molded Product by Dry Method

A 225 ml glass bottle in which 20 g of test piece were sealed was left to stand in a constant temperature bath of 40° C. for 2 hours, and cooled to room temperature. Immediately after that, the odor intensity was rated by ten panelists. The rating criteria used by the panelists were as follows: zero point, no unpleasant odor; 1 point, slightly unpleasant odor; 2 points, distinct unpleasant odor; 3 points, strong unpleasant odor. Total value of the points determined by the ten panelists was used for evaluation.

### Comparative Example 8

Evaluation was conducted in the same manner as in Example 39, except that Me-DBS not containing 12-hydroxystearic acid was used. The results are shown in Table 6.

### Comparative Example 9

Evaluation was conducted in the same manner as in Example 39, except that no nucleating agent was added. The results are shown in Table 6.

### Examples 41 and 42

Evaluation was conducted in the same manner as in Examples 39 and 40, except that a high density polyethylene resin (density=0.967 g/cm3, MFR=6.7 g/10 minutes, hereinafter referred to as “HDPE”) was used as a resin. The results are shown in Table 5.

### Comparative Example 10

Evaluation was conducted in the same manner as in Example 41, except that Me-DBS not containing 12-hydroxystearic acid was used. The results are shown in Table 6.

### Comparative Example 9

Evaluation was conducted in the same manner as in Example 41, except that no nucleating agent was added. The results are shown in Table 6.

TABLE 5

| | | | | | | | | | Odor evaluation | | | | |
|---|---|---|---|---|---|---|---|---|---|---|---|---|---|
| | | Diacetal composition | | | | | | | | Injection-molded | | | |
| | | Com- | | | | | Crystallization | Haze | Pellet | product | | Aldehyde | Taste |
| | | ponent | Component | Amount | Component | Amount | temp. | value | Dry | Dry | Wet | generation | evalu- |
| Ex. | Resin | (A) | (B) | (wt. %) | (C) | (wt. %) | (° C.) | (%) | method | method | method | (μg/PPg) | ation |
| 37 | h-PP | Me- | 12-hydroxystearic | 2.5 | — | — | 133 | 21 | 7 | 9 | 9 | 4.2 | 8 |
| | | DBS | acid | | | | | | | | | | |
| 38 | h-PP | Me- | 12-hydroxystearic | 2.5 | Na lauryl | 2.5 | 133 | 21 | 5 | 5 | 5 | 3.3 | 5 |
| | | DBS | acid | | sulfate | | | | | | | | |
| 39 | LLDPE | Me- | 12-hydroxystearic | 2.5 | — | — | 112 | 30 | 7 | 8 | — | — | — |
| | | DBS | acid | | | | | | | | | | |
| 40 | LLDPE | Me- | 12-hydroxystearic | 2.5 | Na lauryl | 2.5 | 111 | 29 | 6 | 5 | — | — | — |
| | | DBS | acid | | sulfate | | | | | | | | |
| 41 | HDPE | Me- | 12-hydroxystearic | 2.5 | — | — | 117 | — | 6 | 7 | — | — | — |
| | | DBS | acid | | | | | | | | | | |
| 42 | HDPE | Me- | 12-hydroxystearic | 2.5 | Na lauryl | 2.5 | 116 | — | 5 | 5 | — | — | — |
| | | DBS | acid | | sulfate | | | | | | | | |

TABLE 6

| | | | | | | | | | Odor evaluation | | | | |
|---|---|---|---|---|---|---|---|---|---|---|---|---|---|
| | | Diacetal composition | | | | | | | | Injection-molded | | | |
| | | | Com- | | | | Crystallization | Haze | Pellet | product | | Aldehyde | Taste |
| Comp. | | Component | ponent | Amount | Component | Amount | temp. | value | Dry | Dry | Wet | generation | evalu- |
| Ex. | Resin | (A) | (B) | (wt. %) | (C) | (wt. %) | (° C.) | (%) | method | method | method | (μg/PPg) | ation |
| 1 | r-PP | Me-DBS | — | — | — | — | 128 | 11 | 19 | 20 | 19 | 6.2 | 20 |
| 2 | r-PP | DBS | — | — | — | — | 119 | 21 | 20 | 17 | 16 | 6.0 | 19 |
| 3 | r-PP | Et-DBS | — | — | — | — | 127 | 18 | 18 | 18 | 17 | 5.9 | 19 |
| 4 | r-PP | 3,4-DMDBS | — | — | — | — | 128 | 12 | 16 | 16 | 15 | 5.5 | 17 |
| 5 | r-PP | — | — | — | — | — | 104 | 71 | 5 | 5 | 4 | — | — |
| 6 | h-PP | Me-DBS | — | — | — | — | 133 | 20 | 19 | 19 | 18 | 6.3 | 20 |
| 7 | h-PP | — | — | — | — | — | 113 | 74 | 4 | 4 | 3 | — | — |
| 8 | LLDPE | Me-DBS | — | — | — | — | 112 | 29 | 18 | 17 | — | — | — |
| 9 | LLDPE | — | — | — | — | — | 104 | 53 | 4 | 4 | — | — | — |
| 10 | HDPE | Me-DBS | — | — | — | — | 118 | — | 16 | 17 | — | — | — |
| 11 | HDPE | — | — | — | — | — | 112 | — | 3 | 3 | — | — | — |

### INDUSTRIAL APPLICABILITY

When the diacetal composition of the present invention is used as a polyolefin resin nucleating agent, it can provide a polyolefin resin molded product with good clarity in which aldehyde generation is significantly suppressed during the molding process and in the final molded product, whereby transfer of odor and taste is suppressed.

1. An agent for suppressing transfer of odor and taste originating from (A) a diacetal represented by the formula (1): 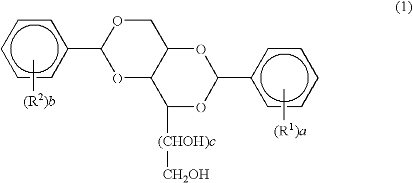
wherein R¹ and R² are the same or different and each represents a hydrogen atom, a C₁ to C₄ alkyl group, a C₁ to C₄ alkoxy group, a C₁ to C₄ alkoxycarbonyl group or a halogen atom; a and b each represents an integer of 1 to 5; c is 0 or 1; when a is 2, the two R¹ groups taken together with the benzene ring to which they are linked may form a tetralin ring; and when b is 2, the two R² groups taken together with the benzene ring to which they are linked may form a tetralin ring;
the agent comprising component (B), i.e., at least one member selected from the group consisting of:
(B₁) C₆ to C₃₂ saturated or unsaturated aliphatic alcohols; and
(B2) C₈ to C₃₂ saturated or unsaturated aliphatic carboxylic acids having at least one hydroxyl group per molecule.

2. The agent for suppressing transfer of odor and taste according to claim 1, wherein said at least one member selected from the group consisting of (B1) and (B2) is at least one member selected from the group consisting of 9-hydroxystearic acid, 10-hydroxystearic acid, 12-hydroxystearic acid, 9,10-dihydroxystearic acid, lauryl alcohol, myristyl alcohol, palmityl alcohol, stearyl alcohol and behenyl alcohol.

3. A method for suppressing aldehyde generation by thermal decomposition of the diacetal represented by the formula (1) according to claim 1;
the method comprising adding to the diacetal represented by the formula (1) at least one member selected from the group consisting of:
(B1) C₆ to C₃₂ saturated or unsaturated aliphatic alcohols; and
(B2) C₈ to C₃₂ saturated or unsaturated aliphatic carboxylic acids having at least one hydroxyl group per molecule.

4. The method according to claim 3, wherein said at least one member selected from the group consisting of components (B1) and (B2) is at least one member selected from the group consisting of 9-hydroxystearic acid, 10-hydroxystearic acid, 12-hydroxystearic acid, 9,10-dihydroxystearic acid, lauryl alcohol, myristyl alcohol, palmityl alcohol, stearyl alcohol and behenyl alcohol.

5. An agent for suppressing transfer of odor and taste originating from (A) at least one diacetal represented by the formula (1): 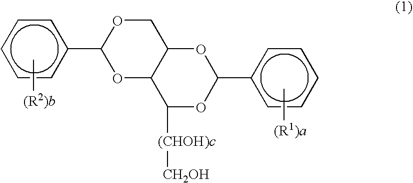
wherein R¹ and R² are the same or different and each represents a hydrogen atom, a C₁ to C₄ alkyl group, a C₁ to C₄ alkoxy group, a C₁ to C₄ alkoxycarbonyl group or a halogen atom; a and b each represents an integer of 1 to 5; c is 0 or 1; when a is 2, the two R¹ groups taken together with the benzene ring to which they are linked may form a tetralin ring; and when b is 2, the two R² groups taken together with the benzene ring to which they are linked may form a tetralin ring;
the agent comprising components (B) and (C),
wherein component (B) is at least one member selected from the group consisting of:
(B1) C₆ to C₃₂ saturated or unsaturated aliphatic alcohols; and
(B2) C₈ to C₃₂ saturated or unsaturated aliphatic carboxylic acids having at least one hydroxyl group per molecule, and
component (C) is
(C₁) at least one anionic surfactant selected from the group consisting of C₆ to C₃₀ saturated or unsaturated aliphatic alcohol sulfuric ester salts, polyoxyethylene alkyl (C₈ to C₂₂) or alkenyl (C₈ to C₂₂) ether sulfuric ester salts in which the number of moles of ethylene oxide a dded is 1 to 8, polyoxyethylene alkyl (C₈ to C₂₂) phenyl ether sulfuric ester salts in which the number of moles of ethylene oxide added is 1 to 10, sulfuric ester salts of polyhydric alcohol fatty acid partial esters formed from a C₃ to C₆ polyhydric alcohol and a C₈ to C₂₂ saturated or unsaturated fatty acid, and C₈ to C₂₂ saturated or unsaturated fatty acid monoalkanol (C₂ to C₆) amide sulfuric ester salts, wherein the sulfuric ester salts are lithium salts, sodium salts, potassium salts and ammonium salts;
(C2) at least one member selected from the group consisting of alkali metal salts of C₈ to C₃₂ saturated or unsaturated fatty acids which may have at least one hydroxyl group per molecule;
(C3) at least one aliphatic amine selected from the group consisting of dialkanolamine, trialkanolamine, and di(C₈ to C₂₂ alkyl or alkenyl) methylamine; or
(C4) a mixture of at least two of (C1), (C2) and (C3).

6. The agent for suppressing transfer of odor and taste according to claim 5, wherein
component (B) is at least one member selected from the group consisting of 9-hydroxystearic acid, 10-hydroxystearic acid, 12-hydroxystearic acid, 9,10-dihydroxystearic acid, lauryl alcohol, myristyl alcohol, palmityl alcohol, stearyl alcohol and behenyl alcohol, and
component (C) is (C2a) at least one member selected from the group consisting of lithium salts, sodium salts and potassium salts of C₈ to C₃₂ saturated or unsaturated fatty acids which may have at least one hydroxyl group per molecule, or
component (C) is (C I a) at least one sulfuric ester salt selected from the group consisting of lauryl sulfate salts, stearyl sulfate salts, oleyl sulfate salts, polyoxyethylene (the number of moles of ethylene oxide added=2 to 3) lauryl ether sulfate salts, polyoxyethylene (the number of moles of ethylene oxide added=2 to 3) stearyl ether sulfate salts, polyoxyethylene (the number of moles of ethylene oxide added=2 to 3) nonylphenyl ether sulfate salts, polyoxyethylene (the number of moles of ethylene oxide added=2 to 3) dodecylphenyl ether sulfate salts, glyceryl monolaurate sulfate salts, glyceryl monostearate sulfate salts, lauric acid monoethanolamide sulfuric ester salts, stearic acid monoethanolamide sulfuric ester salts, and oleic acid monoethanolamide sulfuric ester salts, wherein the sulfuric ester salts or sulfate salts are lithium salts, sodium salts and potassium salts.

7. The agent for suppressing transfer of odor and taste according to claim 6, wherein component (C) is at least one member selected from the group consisting of sodium lauryl sulfate, potassium lauryl sulfate, sodium stearate, potassium stearate, sodium 12-hydroxystearate and potassium 12-hydroxystearate.

8. The agent for suppressing transfer of odor and taste according to claim 5, wherein the weight ratio of component (B) to component (C) is 1:0.2 to 5.

9. A method for suppressing aldehyde generation by thermal decomposition of (A) at least one diacetal represented by the formula (1): 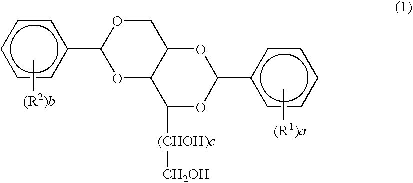
wherein R¹ and R² are the same or different and each represents a hydrogen atom, a C₁ to C₄ alkyl group, a C₁ to C₄ alkoxy group, a C₁ to C₄ alkoxycarbonyl group or a halogen atom; a and b each represents an integer of 1 to 5; c is 0 or 1; when a is 2, the two R¹ groups taken together with the benzene ring to which they are linked may form a tetralin ring; and when b is 2, the two R² groups taken together with the benzene ring to which they are linked may form a tetralin ring;
the method comprising adding the following components (B) and (C) to the diacetal, wherein component (B) is at least one member selected from the group consisting of:
(B1) C₆ to C₃₂ saturated or unsaturated aliphatic alcohols; and
(B2) C₈ to C₃₂ saturated or unsaturated aliphatic carboxylic acids having at least one hydroxyl group per molecule, and
component (C) is
(C1) at least one anionic surfactant selected from the group consisting of C₆ to C₃₀ saturated or unsaturated aliphatic alcohol sulfuric ester salts, polyoxyethylene alkyl (C₈ to C₂₂) or alkenyl (C₈ to C₂₂) ether sulfuric ester salts in which the number of moles of ethylene oxide a dded is 1 to 8, polyoxyethylene alkyl (C₈ to C₂₂) phenyl ether sulfuric ester salts in which the number of moles of ethylene oxide added is 1 to 10, sulfuric ester salts of polyhydric alcohol fatty acid partial esters formed from a C₃ to C₆ polyhydric alcohol and a C₈ to C₂₂ saturated or unsaturated fatty acid, and C₈ to C₂₂ saturated or unsaturated fatty acid monoalkanol (C₂ to C₆) amide sulfuric ester salts, wherein the sulfuric ester salts are lithium salts, sodium salts, potassium salts and ammonium salts;
(C2) at least one member selected from the group consisting of alkali metal salts of C₈ to C₃₂ saturated or unsaturated fatty acids which may have at least one hydroxyl group per molecule;
(C3) at least one aliphatic amine selected from the group consisting of dialkanolamine, trialkanolamine, and di(C₈ to C₂₂ alkyl or alkenyl) methylamine; or
(C4) a mixture of at least two of (C1), (C2) and (C3).

10. The method according to claim 9, wherein component (B) is at least one member selected from the group consisting of 9-hydroxystearic acid, 10-hydroxystearic acid, 12-hydroxystearic acid, 9,10-dihydroxystearic acid, lauryl alcohol, myristyl alcohol, palmityl alcohol, stearyl alcohol and behenyl alcohol, and
component (C) is (C2a) at least one member selected from the group consisting of lithium salts, sodium salts and potassium salts of C₈ to C₃₂ saturated or unsaturated fatty acids which may have at least one hydroxyl group per molecule, or
component (C) is (C1a) at least one sulfuric ester salt selected from the group consisting of lauryl sulfate salts, stearyl sulfate salts, oleyl sulfate salts, polyoxyethylene (the number of moles of ethylene oxide added=2 to 3) lauryl ether sulfate salts, polyoxyethylene (the number of moles of ethylene oxide added=2 to 3) stearyl ether sulfate salts, polyoxyethylene (the number of moles of ethylene oxide added=2 to 3) nonylphenyl ether sulfate salts, polyoxyethylene (the number of moles of ethylene oxide added=2 to 3) dodecylphenyl ether sulfate salts, glyceryl monolaurate sulfate salts, glyceryl monostearate sulfate salts, lauric acid monoethanolamide sulfuric ester salts, stearic acid monoethanolamide sulfuric ester salts, and oleic acid monoethanolamide sulfuric ester salts, wherein the sulfuric ester salts or sulfate salts are lithium salts, sodium salts and potassium salts.

11. The method according to claim 10, wherein component (C) is at least one member selected from the group consisting of sodium lauryl sulfate, potassium lauryl sulfate, sodium stearate, potassium stearate, sodium 12-hydroxystearate and potassium 12-hydroxystearate.

12. The method according to claim 9, wherein the weight ratio of component (B) to component (C) is 1:0.2 to 5.

13. A granular or powdery diacetal composition comprising:
(A) at least one diacetal represented by the formula (1) 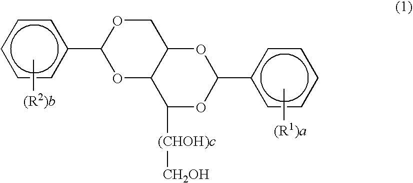
wherein R¹ and R² are the same or different and each represents a hydrogen atom, a C₁ to C₄ alkyl group, a C₁ to C₄ alkoxy group, a C₁ to C₄ alkoxycarbonyl group or a halogen atom; a and b each represents an integer of 1 to 5; c is 0 or 1; when a is 2, the two R¹ groups taken together with the benzene ring to which they are linked may form a tetralin ring; and when b is 2, the two R² groups taken together with the benzene ring to which they are linked may form a tetralin ring; and
component (B), i.e., at least one member selected from the group consisting of
(B1) C₆ to C₃₂ saturated or unsaturated aliphatic alcohols and
(B2) C₈ to C₃₂ saturated or unsaturated aliphatic carboxylic acids having at least one hydroxyl group per molecule,
wherein transfer of odor and taste originating from the diacetal is suppressed.

14. The diacetal composition according to claim 13, wherein component (B) is present in a proportion of 0.1 to 10 wt % based on the total amount of components (A) and (B).

15. A granular or powdery diacetal composition wherein transfer of odor and taste originating from the diacetal is suppressed;
the composition comprising components (A), (B) and (C),
wherein component (A) is at least one diacetal represented by the formula (1) 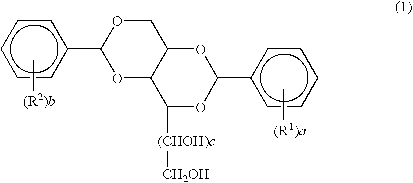
wherein R¹ and R² are the same or different and each represents a hydrogen atom, a C₁ to C₄ alkyl group, a C₁ to C₄ alkoxy group, a C₁ to C₄ alkoxycarbonyl group or a halogen atom; a and b each represents an integer of 1 to 5; c is 0 or 1; when a is 2, the two R¹ groups taken together with the benzene ring to which they are linked may form a tetralin ring; and when b is 2, the two R² groups taken together with the benzene ring to which they are linked may form a tetralin ring, component (B) is at least one member selected from the group consisting of:
(B1) C₆ to C₃₂ saturated or unsaturated aliphatic alcohols; and
(B2) C₈ to C₃₂ saturated or unsaturated aliphatic carboxylic acids having at least one hydroxyl group per molecule, and
component (C) is
(C₁) at least one anionic surfactant selected from the group consisting of C₆ to C₃₀ saturated or unsaturated aliphatic alcohol sulfuric ester salts, polyoxyethylene alkyl (C₈ to C₂₂) or alkenyl (C₈ to C₂₂) ether sulfuric ester salts in which the number of moles of ethylene oxide a dded is 1 to 8, polyoxyethylene alkyl (C₈ to C₂₂) phenyl ether sulfuric ester salts in which the number of moles of ethylene oxide added is 1 to 10, sulfuric ester salts of polyhydric alcohol fatty acid partial esters formed from a C₃ to C₆ polyhydric alcohol and a C₈ to C₂₂ saturated or unsaturated fatty acid, and C₈ to C₂₂ saturated or unsaturated fatty acid monoalkanol (C₂ to C₆) amide sulfuric ester salts, wherein the sulfuric ester salts are lithium salts, sodium salts, potassium salts and ammonium salts;
(C2) at least one member selected from the group consisting of alkali metal salts of C₈ to C₃₂ saturated or unsaturated fatty acids which may have at least one hydroxyl group per molecule;
(C3) at least one aliphatic amine selected from the group consisting of dialkanolamine, trialkanolamine, and di(C₈ to C₂₂ alkyl or alkenyl) methylamine; or
(C4) a mixture of at least two of (C1), (C2) and (C3).

16. The diacetal composition according to claim 15, wherein based on the total amount of components (A), (B) and (C), component (B) is present in a proportion of 0.1 to 5 wt % and component (C) is present in a proportion of 0.1 to 5 wt %.

17. The diacetal composition according to claim 16, wherein the weight ratio of component (B) to component (C) is 1:0.2 to 5.

18. A polyolefin resin nucleating agent comprising the diacetal composition according to claim 13, wherein transfer of odor and taste originating from the diacetal is suppressed.

19. A polyolefin resin composition comprising the polyolefin resin nucleating agent according to claim 18 and a polyolefin resin, wherein transfer of odor and taste originating from the diacetal is suppressed.

20. The polyolefin resin composition wherein the polyolefin resin nucleating agent according to claim 18 is present in an amount of 0.05 to 3 weight parts per 100 weight parts of the polyolefin resin and wherein transfer of odor and taste originating from the diacetal is suppressed.

21. A polyolefin resin molded product prepared by molding the polyolefin resin composition according to claim 19, wherein transfer of odor and taste originating from the diacetal is suppressed.

22. A container or a packaging material for foods, cosmetics or medicines comprising the polyolefin resin molded product according to claim 21, wherein transfer of odor and taste originating from the diacetal is suppressed.

23. A method for suppressing odor originating from a diacetal at the time of molding a polyolefin resin, comprising mixing the nucleating agent according to claim 18 with a polyolefin resin and molding a resultant resin composition.

24. A method for suppressing transfer of odor and taste originating from a diacetal to a content (such as foods, cosmetics and medicines), characterized in that it comprises placing the content in a packaging material or a container prepared by mixing the nucleating agent according to claim 18 with a polyolefin resin and molding a resultant resin composition.
